(19) United States
(12) Patent Application Publication
(10) Pub. No.: US 2021/0215584 A1
(43) Pub. Date: Jul. 15, 2021

(54) DEVICES, METHODS, AND KITS FOR SAMPLE ANALYSIS USING MICROSLIT FILTERS

(71) Applicant: SIMPORE INC., West Henrietta, NY (US)

(72) Inventors: Jared A. Carter, Rochester, NY (US); James A. Roussie, Rochester, NY (US)

(73) Assignee: SIMPORE INC., West Henrietta, NY (US)

(21) Appl. No.: 17/268,330

(22) PCT Filed: Aug. 16, 2019
(86) PCT No.: PCT/US2019/046947

§ 371 Date: Feb. 12, 2021

Related U.S. Application Data
(60) Provisional application No. 62/719,013, filed on Aug. 16, 2018.
(60) Provisional application No. 62/754,946, filed on Nov. 2, 2018.

(51) Int. Cl. G01N 1/40; G01N 33/50; B01D 61/18; B01D 61/14; B01D 69/02; B01D 71/02

(52) U.S. Cl. CPC G01N 1/4077; G01N 33/5094; B01D 61/18; B01D 61/147; B01D 69/02; B01D 2315/10; G01N 2001/4088; B01D 2325/021; B01D 2325/04; B01D 2317/04; B01D 2317/06; B01D 71/02

(57) ABSTRACT

Provided are methods, devices, and kits for the isolation and enumeration of one or more components of interest within a liquid sample using microslit filter membranes. This disclosure relates to the enumeration of components within a sample of interest, and more particularly, the capture of such components by efficient isolation using microslit filters with high permeation capacity and precision molecular cut-off characteristics.

### CROSS REFERENCE TO RELATED APPLICATIONS

This application claims priority to U.S. Provisional Application No. 62/719,013, filed on Aug. 16, 2018, and to U.S. Provisional Application No. 62/754,946, filed on Nov. 2, 2018, the disclosures of which are incorporated by reference herein.

### FIELD OF THE DISCLOSURE

This disclosure relates to the enumeration of components within a sample of interest, and more particularly, the capture of such components by efficient isolation using microslit filters with high permeation capacity and precision molecular cut-off characteristics.

### BACKGROUND OF THE DISCLOSURE

The enumeration of a desired species of interest, such as, for example, cells or other components, within a liquid sample is a critical step required for research and clinical analytical purposes. Enumeration may comprise the counting of, or determining concentration of, such components in a liquid sample.

While many methods have been developed for these purposes, the most widely used method involves passing the species of interest through a single aperture for its enumeration, wherein electrical signals are used to count species passage events (in order to determine concentration) and to size the species as well.

One well-known problem associated with single aperture component counters is their tendency to clog (e.g., become irreversibly filled with species so that they no longer pass any additional species). This clogging phenomenon is often due to the complexity of the fluid matrix and the highly abundant concentration of species in the fluid samples often counted by the single aperture method. For example, complete blood cell counts (CBC) are performed by single aperture counters. However, the high abundance of hemocytes, as well as matrix factors (e.g., serum albumin), can clog the single aperture, distort its readings, and lead to down-time on the counter instrument. Similar problems are encountered when assessing the concentration of components in samples such as, for example, chemical products or industrial effluents or discharges, where a fluid sample is tested for concentration and size of particulate matter.

The problem associated with single aperture-based approaches is their limited capture area for species of interest. For purposes of this disclosure, it is understood that capture area relates to the amount of surface area or volume in which the species of interest are captured. Since the single aperture has a limited volume in which species of interest are captured for enumeration, its clogging problems can be associated with this aspect.

What is needed is a method for the enumeration of components that is not prone to clogging as are single aperture-based methods.

### BRIEF DESCRIPTION OF THE FIGURES

For a fuller understanding of the nature and objects of the disclosure, reference should be made to the following detailed description taken in conjunction with the accompanying figures.

FIG. 1 provides images of a representative microslit filter (B) and a representative fluidic device (A) incorporating such a microslit filter.

FIG. 2 provides exemplary uses (A, B, C) of microslit filters for retaining and measuring biological and non-biological components from liquid samples.

FIG. 3 provides exemplary uses (A, B, C, D) of microslit filters for retaining and measuring biological and non-biological components from liquid samples.

FIG. 4 provides an example of a microslit filter with an underlying fluidic cavity (A), an example of a scheme for fluidically connecting multiple filtration membranes and intervening fluidic cavities (B), and an example of a fluidic rendering of a device for fluidically addressing a membrane system.

FIG. 5 show panels (A, B, C, D, E, F) of images describing the design and modeling of an exemplary tangential flow fluidic device for incorporating microslit filters.

FIG. 6 demonstrates the results (A, B, C) of a pressure drop analysis which evaluated microslit height.

FIG. 7 shows resulting phase contrast microscopy images (A, B, C, D).

FIG. 8 shows the results (A, C, D) from the selective retention by a microslit filter of a polymeric particle, and exemplary devices (B) of the present disclosure.

FIG. 9 shows particle retention and permeation results (A) for three different nanoporous filters and a representative 100 nm-thick nanoporous filter (B) of the present disclosure.

FIG. 10 shows particle retention and permeation results for two different microslit filters.

FIG. 11 provides micrographic images (A, B) of microslit filters of the present disclosure.

FIG. 12 shows particle retention and permeation results for a microslit filter of the present disclosure.

FIG. 13 shows (A) transmission of various nanoparticle sizes through a microslit filter and a representative polymeric filter, and a graph (B) showing the difference in fouling between a microslit filter and a representative polymeric filter.

FIG. 14 shows (A, B) a microslit filter of the present disclosure.

FIG. 15 shows a phase contrast light microscopy image (A) of a microslit filter, and the same microslit filter showing an IR signature of microplastic particles retained on the membrane surface (B).

FIG. 16 provides an exemplary ex situ analytical assay of the present disclosure, wherein infrared spectroscopy was used to identify the composition of a first type of particle (e.g., poly (methyl methacrylate)—PMMA) retained by a microslit filter.

FIG. 17 provides an exemplary ex situ analytical assay (A, B, C) of the present disclosure, wherein infrared spectroscopy was used to identify the composition of a second type of particle (e.g., polyethylene) retained by a microslit filter.

FIG. 18 provides (A, B, C) an exemplary use of a transmembrane pressure-dependent method of the present disclosure for a first type of biological component (e.g., Adenovirus) and compares the method between a microslit filter and a representative polymeric filter.

FIG. 19 provides (A, B) an exemplary use of a transmembrane pressure-dependent method of the present disclosure for a second type of biological component (e.g., Maraba virus) and compares the method between a microslit filter and a representative polymeric filter.

FIG. 20 provides an exemplary use of a transmembrane pressure-dependent method of the present disclosure for a third type of biological component (e.g., B. diminuta bacteria) and compares the method between a microslit filter and a representative polymeric filter.

FIG. 21 provides micrographic images and physical dimensions of representative nanoporous and microslit filters of the present disclosure.

FIG. 22 provides various membrane physical properties of nanoporous and microslit filters of the present disclosure.

FIG. 23 provides micrographic images (A, B, C, D) of microslit and microporous filters of the present disclosure.

FIG. 24 provides micrographic images (A, B, C) of microporous filters of the present disclosure.

FIG. 25 provides micrographic images (A, B, C, D, E, F) of nanoporous filters of the present disclosure.

FIG. 26 provides micrographic images of microslit filters of the present disclosure.

### DETAILED DESCRIPTION OF THE DISCLOSURE

Although claimed subject matter will be described in terms of certain embodiments and examples, other embodiments and examples, including embodiments and examples that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

In an aspect, methods for the enumeration (e.g., quantifying or counting of or determining concentration of) one or more species of interest (e.g., one or more component of interest) from a liquid sample are disclosed. The methods are based on capture and retention of such components by microslit filters of the present disclosure.

In an example, a method of the present disclosure comprises filtering the sample to capture and retain the desired components, while removing or permeating undesired components, optionally, washing the captured components to remove any non-specifically captured components, and determining (e.g., measuring) the number of captured and retained components to determine their concentration in the liquid sample. The filtration that captures and retains desired components from undesired solutes makes use of a filtration membrane of specified characteristics. This filtration membrane can be referred to as a microslit filter or as microslit filters. Such microslit filters cam comprise cubic or rectangular prism openings of specified width, length, and height (e.g., aspect ratio) within a freestanding membrane.

In an example, a method for enumerating components of interest from a liquid sample comprises:
a) contacting the liquid sample with a microslit filter, such that the components of interest are retained by the microslit filter and undesired components permeate through the microslit filter;
b) optionally, washing the retained components; and
c) measuring the quantity of retained components to determine their concentration in the liquid sample.

Components of interest may include, but are not limited to, biological species (i.e., biological components) such as, for example, intact cells, sub cellular components (e.g., vesicles, organelles, and the like), macromolecular complexes (e.g., protein-nucleic acid, lipoprotein complexes, and the like), viruses, bacteria, fungi, protozoa, and the like, and any combinations of any of these species.

The liquid sample bearing components of interest (e.g., biological components of interest) may be a biofluid, which may include, but are not limited to, cell lysates, venous or arterial whole blood, plasma, serum, sputum, urine, cerebrospinal fluid, conditioned cell culture media or other fluids derived from cell culture and other fluids containing molecules of biological origin, or any solutions in contact with biological tissues (such as, for example, bodily secretions, discharges, and/or excretions, as well as swabs and/or aspirates of bodily tissues, among others,), or any combination thereof. As one example of conditioned cell culture media or other fluids derived from cell culture (e.g., a cell lysate) that bear the biological components of interest (e.g., a cell, a protein, a virus, or an extracellular vesicle, among others), the culture may be an adherent or a suspension mammalian cell culture that is used to produce the biological components, and one or more of the methods disclosed herein is used to evaluate the production of such biological components in the culture. For example, the culture may be a suspension culture of genetically modified Chinese hamster ovary cells secreting humanized monoclonal antibody. In another example, the culture may be a suspension culture of human embryonic kidney cells genetically modified to produce a lenti virus vector. In another example, the culture may be a suspension culture of genetically modified T-cells for adoptive cell transfer therapy (e.g., chimeric antigen receptor T-cell therapy). In another example, the culture may be an adherent culture of autonomous mesenchymal stem cells secreting extracellular vesicles for therapeutic applications. In some examples, an optional pretreatment (i.e., sample pretreatment) of the biofluid sample may be carried out prior to carrying out the methods of the present disclosure, such as, for example, dilution of the liquid sample in an appropriate buffer or solution compatible the nature of the liquid sample, low-speed centrifugation of whole blood to remove hemocytes (thus forming a plasma sample), lysis of a population of cells (thus forming a cell lysate), chemical or enzymatic digestion of biologically derived materials (thus forming a sample of components resistant to such digestion), or fluidization of a solid sample (thus forming a liquid sample) among many other possible pretreatment alternatives. In other examples, sample pretreatment may comprise filtration through a microslit filter to retain certain species, while permeating other species.

Other components of interest may include, but are not limited to, non-biological species (i.e., non-biological components) such as, for example, nano- or micro-components of organic, polymeric, inorganic, or mixed composition, organic compounds, complexes or aggregates, inorganic salts or aggregates, and the like, and any combinations of any of these. Other examples of components of interest include, but are not limited to, gold, silver, silica, agarose, polystyrene, latex, iron oxide, cerium oxide, cadmium selenide, among others. Other examples of components of interest may further include, but are not limited to, acrylonitrile butadiene styrene, polyester, polyethylene, polymethylmethacrylate, polyethylene terephthalate, polyvinyl chloride, polypropylene, polylactic acid, polycarbonate, polyoxymethylene, aliphatic- and aromatic-substituted polyamides or polyimines, polytetrafluoroethylene, or per- and poly-fluoroalkyl polymers, polyketones, polyglycols, polypeptides, among other polymers. Other examples of components of interest may further include, but are not limited to, silicon-based or carbon-based fibers. Of course, other possible types of non-biological components are possible and these examples have been provided merely for exemplary purposes.

Non-biological liquid samples that are compatible with the present disclosure include, but are not limited to, samples of water, industrial discharges, food products, milk, air filtrates, among others, and thus include, but are not limited to, food, environmental and industrial samples.

For purposes of this disclosure, it is understood that a plurality of desired components is retained by the microslit filter, while a plurality of undesired components permeates the microslit filter, upon the filtration step (e.g., of a)).

In various examples, the liquid sample can bear populations of both biological components and non-biological components. The methods disclosed herein (e.g., steps a) through c)), in combination with any optional pre-treatment step, can be used to measure the concentration of the one or more populations of biological and non-biological components, and further, the one or more analytical assays described hereunder can be optionally used to determine the composition of such populations of both components.

The filtration (e.g., of a) can be performed by contacting the liquid sample with a microslit filter of specified characteristics. For example, the width of the microslit filter's openings can be specified to retain desired components of a certain diameter and permeate other undesired components of another diameter. As one example, the microslit filter can have either cubic prism or rectangular prism openings, where the width of such prism-like openings is specified relative to the diameter of spherical components of interest in the liquid sample. Accordingly, the width of the microslit filter's openings must be specified in order to affect the desired retention or permeation outcomes.

To retain a desired component of a certain diameter, the retained components' diameter must be greater than the width of the microslit filter's openings. Accordingly, it is recognized that retention of such components is desired as an isolation for their subsequent enumeration by the methods disclosed herein. By contrast, to permeate an undesired component of another diameter (e.g., smaller diameter), the permeated components' diameter must be less than the width of the microslit filter's openings. As one example, a microslit filter may be specified to capture one cell type from whole blood (e.g., leukocytes and the like), while permeating other cell types (e.g., erythrocytes, platelets, and the like), relying on the relative size differences between larger leukocytes over platelets and erythrocytes, in order to retain the former species and permeate the latter two species. As another example, a microslit filter can be specified to capture polymeric components of a certain diameter (and larger) out of the effluent of a waste water treatment plant and to permeate other smaller species in the effluent, such that the retained polymeric components are likely to comprise a range of component diameter that are equal and greater than the opening width of the microslit filter used for the filtration. In both examples, capture and retention of either the leukocytes or polymeric components is desired so that the number of such species can be determined from a known volume of the liquid sample. Of course, other component types can be captured and retained by the methods disclosed herein and these examples have been provided merely for exemplary purposes.

The filtration (e.g., of a)) can be performed by several flow modalities. For example, the filtration comprises dead-end or normal flow initiated by gravity, hydrostatic pressure, pumping, vacuum, centrifugation, or gas pressurization, wherein the liquid sample is introduced to the cis-side of the microslit filter and translocates to the opposing trans-side of the microslit filter. In another example, the filtration comprises tangential flow initiated by liquid sample alone or in combination with a buffer solution, respectively, on opposing cis and trans-sides of the microslit filter. The tangential flow modality may further comprise diafiltration, wherein a specified transmembrane pressure vector drives both capture/retention of desired components of interest and permeation of undesired components through the microslit filter, while a specified tangential flow vector parallel to the microslit filter surface drives downstream flow of non-captured/retained components within the openings of the microslit filter (thereby reducing clogging or fouling). It should be recognized that, in examples wherein tangential flow is used, some proportion the larger, desired components will be swept downstream by the tangential flow vector parallel to the microslit filter surface and that some proportion of the smaller, undesired components will be similarly swept downstream (and not permeate through the microslit filter).

In some examples, one or more washing steps can follow a) in order to remove nonspecifically and undesired components associated with the retained components of interest. The wash solution may be any buffer or solution appropriate to the nature and composition of the components of interest and to that of the matrix components of the liquid sample. For biological components, as an example, the wash solution may include, but is not limited to, ionic or non-ionic detergents or have specified salt concentration or pH to promote removal of non-specifically bound and undesired components. For example, such washing steps using a normal saline buffer (e.g., phosphate-buffered saline and the like) may be used to remove any erythrocytes and/or platelets nonspecifically associated with retained leukocytes, by washing the retained leukocytes in a bolus of such buffer and then performing the filtration a second time. Other buffers, solutions, and the like may be more appropriate for non-biological components (e.g., polar or non-polar solvents). Of course, other washing conditions are possible and these examples have been provided merely for exemplary purposes. Similar flow modalities as described for the filtration of a) may be used for any washing steps. Any such washing steps, if included, may remove smaller, non-desired components and species (e.g., matrix components and the like) from any retained components and such non-desired components or species may be transferred to a waste chamber or vessel as part of any washing steps. Moreover, if such washing steps are used in a tangential flow configuration, they may promote removal of non-specifically bound undesired components by both permeation and downstream flow.

In some examples, an optional upstream sample preparation step may be additionally used. In addition to the examples previously described, a liquid sample may be contacted by a first microslit filter, wherein the openings in this first microslit filter are specified to reject undesired components (e.g., components with a diameter larger than the rectangular prism openings of a microslit filter), and to permeate the desired components of interest, which are then subsequently contacted by a second microslit filter (e.g., the microslit filter of a)). In these examples, the outflow of the first microslit filter is in fluid contact with the second microslit filter, such that the permeate of the first filter can be contacted by the second microslit filter.

In the various examples of the methods disclosed herein, the microslit filter-based format may overcome well-known problems of single aperture-based formats for determining the number of components of interest in a liquid sample. For example, a microslit filter offers greater surface area and volume over that of a single aperture, since the microslit filter provides a plurality of openings (e.g., apertures) that do not foul and clog like the limited surface area and volume of a single aperture. If a microslit filter becomes occluded anywhere along the length of its openings, there remains additional surface area and volume along the remaining length of its openings for continued operation. By contrast, once a single aperture becomes clogged with a species, it can no longer permeate additional species and continue its usual mode of operation. In addition, the straight-through, cubic or rectangular prism openings of microslit filters (rather than a cylindrical opening of a single aperture) enables low permeation resistance filtration properties. The low permeation resistance of microslit filters is a consequence of their thickness, aspect ratio of prism-like openings, and minimal internal surface area. This combination of properties endows microslit filters with high filtration capacity; e.g., the ability to permeate a large number of undesired components without clogging. Furthermore, the specified aspect ratio of the prism openings of microslit filters enables selective filtration outcomes; e.g., the ability to retain desired components of interest in a selective manner, while permeating undesired components. Finally, it is recognized that the permeation resistance of microslit filters is proportional to its thickness (e.g., lower membrane thickness provides low permeation resistance), but is inversely proportional to its porosity (e.g., higher porosity provides low permeation resistance). Accordingly, the methods of the present disclosure are further enabled by the combination of low thickness (e.g., ≤1 μm membrane thickness) and high porosity (e.g., ≥10%) of the exemplary microslit filters disclosed herein. For purposes of this disclosure, porosity refers to the plurality of openings (e.g., apertures) through the membrane per unit of membrane surface area.

The filtration (e.g., of a)) can be performed at a range of pressurization that is compatible with maintaining the integrity of species in a liquid sample. This compatibility relies on the low permeation resistance property of microslit filters. For example, the pressurization can be from 10 Pa to 1.0 kPa and all Pa values therebetween. In another example, the pressurization can be that only applied by hydrostatic pressure. In some examples, wherein the sample is a biofluid and the components of interest are cells (e.g., hemocytes of whole blood and the like), the pressurization can be sufficiently low that the shear forces applied to cells when permeating the microslit filters do not cause cell lysis. For example, a microslit filter of 8×50 μm openings and 400 nm thickness (membrane thickness) applies a maximum of 18 dynes/cm² at 1.0 kPa, while 1,500 dynes/cm² is known, for example, to cause erythrocyte lysis (Leverett et al, 1972).

To determine the number of captured and retained components on the microslit filter, a known volume of liquid sample is passed via the flow modalities described herein, the number of retained components assessed by the measuring methods described herein, and the concentration of components calculated by dividing the number of retained components by the known volume of liquid sample that was passed. In some examples, a fluidic device is configured to pass a known volume of liquid sample. In another example, a flow meter within a fluidic device measures the volume of passed liquid sample. In various examples, an algorithm is used to record retained components, to tracked volume of liquid sample passed and to calculate component concentration. In some examples, such calculations may be performed on repeat or replicate measurements of the same component of interest; for instance, the component can be captured and retained on multiple microslit filters within one fluidic device, and an algorithm is used to calculate the arithmetic mean of the multiple instances of retained components. In such examples, the reported component concentration is the average of the data collected from the multiple instances. Alternatively, a single instance of measured component retention and volume passage is used to calculate the component concentration, which can be reported as a single datum.

The step of determining the number of retained components (e.g., step c)) by any of the measurement methods disclosed herein can be performed as either in situ or ex situ measurements, with respect to the fluidic devices used for the contacting and optional washing steps (e.g., steps a) and b), respectively). As an example of an in situ measurement, the microslit filter remains within a fully assembled or partially disassembled fluidic device when performing the measurement. In this example, the fluidic device may be transferred into an instrument for carrying out any measurement. In an example of an ex situ measurement, the microslit filter is extracted from a partially or fully disassembled fluidic device and the microslit filter is transferred to an instrument for carrying out any measurement. In this latter example, the extracted microslit filter may be optionally transferred to another substrate prior to transfer into the instrument carrying out the measurement.

The measuring method performed on the retained components (e.g., of c)), to determine the number of retained components, can be carried out by a variety of modalities and methods. For example, the measuring methods can include, but are not limited to, optical imaging, electronic interrogation, and optical diffraction modalities. For purposes of this disclosure, both optical imaging and optical diffraction, as well as other optically dependent first and second analytical assays disclosed hereunder, are considered collectively as optical modalities or as optical methods. Such measurements can be performed in a single instance or as replicate instances and their results reported as a single datum or arithmetic mean values.

The measuring method performed on the retained components (e.g., of c)), to determine the number of retained components, can also be carried out by a transmembrane pressure-dependent modality and method. For example, the measuring methods can include a change or differential measurement for the transmembrane pressure across a microslit filter or microslit membrane. Such measurements can be performed in a single instance or as replicate instances and their results reported as a single datum or arithmetic mean values.

In various examples, the measuring method performed on the retained components (e.g., of c)) to determine their concentration can be a combination of optical imaging, electronic interrogation, optical diffraction, and transmembrane pressure modalities.

for example, the measuring method can be a combination of optical imaging and transmembrane pressure methods. In another example, the measurement can be a combination of optical imaging and electronic interrogation. In various examples, the two or more measurement methods can be performed one after the other (in any temporal order) or may be performed simultaneously. The results from such combined measurements may be reported as single instances, arithmetic means, and/or as correlated data between the two or more measurement methods.

In an example, the step of determining the number of retained components can comprise optical imaging. For example, captured and retained components may be counted using an illuminating light source and a light detector and the number of components counted by an algorithm that processes the resultant images (e.g., a signal processing algorithm). In some examples, a physical property of a particular component type may be used to determine the number of retained components (e.g., fluorescence, luminescence, absorbance, and the like, and combinations thereof). For example, the hemoglobin content within retained erythrocytes may be used as the basis of an absorbance optical signal at a specified wavelength for determining the number of retained erythrocytes. As another example, a fluorescent signal of a cadmium selenide quantum dot, generated at a specified excitation wavelength and recorded at a specified emission wavelength, may be used. A light source and a detector may be used for optical imaging, excitation and recording of emission, luminescent and/or absorbance signals, and may further include a light source and a detector, in a variety of fashions including photodiode arrays, charge coupled devices, and other optical sensing techniques for carrying out the optical signal detection methods. Of course, other physical properties, as well as detection modalities, may be used and these examples have been provided merely for exemplary purposes.

In other examples, the optical imaging can further comprise one or more plasmonic-enhanced optical imaging modality; e.g., surface plasmon resonance, plasmon- or surface-enhanced fluorescence, surface-enhanced Raman spectroscopy, and the like. As known to those skilled in the related art, incident light may plasmonically excite molecules on retained components, and thus upon optical interrogation, amplify emission spectra and improve limit of detection and sensitivity. Further, surface plasmon resonance may rely on a shift in refractive index caused by such plasmon excitation, but may be affected by ambient temperature. In some examples of plasmonic detection, microslit filters may be conformally coated with a noble metal that is plasmonically active (e.g., Au, Pt, Ag, Ir, Rh, and the like, and combinations thereof).

In some examples comprising an optical imaging modality, microslit filters may be conformally coated to reduce any signal contributed by the microslit filter with respect to the optical imaging modality; that is, the coating may reduce background signal and thus improve signal-to-noise (or resolution and sensitivity) of the optical imaging. In some examples, the coating may be a conformal coating and may include, but is not limited to, Ti, Cr, Al, Au, Pt, Ag, Ir, Rh (and the like, as well as various combinations thereof), or MgF₂, CaF₂, TiO₂ (and the like, as well as various combinations thereof). In one example, the optical modality is Fourier-transformed infrared spectroscopy and the microslit filter is coated with a metal (e.g., Al, Au, or Ag) and such metal coating comprises an infrared light reflective layer. In another example, the optical modality is Raman spectroscopy and the coating is a Raman-silent layer (e.g., MgF₂, CaF₂, or TiO₂). The thickness of the coating may be specified to affect the desired property (e.g., infrared reflectivity or Raman silence). Accordingly, the coating may have a range of thickness; for example, the thickness can be from 10 nm to 1,000 nm, including all values to the nm and ranges therebetween. Of course, other combinations of microslit filter coatings and optical modalities are possible and these examples have been provided merely for exemplary purposes.

In various examples, the measurement (e.g., of c)) can further comprise an electronic interrogation modality based on transmembrane electrical resistance or transmembrane impedance, among other possibilities. For example, upon retention of desired components by a microslit filter, the openings of such filters are occluded, such that the trans-membrane electrical resistance to an input current increases or is blocked altogether. One or more pairs of electrodes may be used to measure the transmembrane electrical resistance, if placed on opposing cis- and trans-sides of a microslit filter. As another example, a function generator may be used to generate an input current at high frequency, such that trans-membrane impedance may be recorded. The impedance of an interface is generally determined by applying a sinusoidal voltage perturbation, while simultaneously recording the current response using a voltmeter. A linear voltage-current response may be obtained by small (e.g., ˜10 mV peak to peak) perturbations. Such voltage-current responses thus provide the related transmembrane impedance. One or more pairs of electrodes may be used to measure such transmembrane impedance, in connection with a function generator and voltmeter, if the electrodes placed on opposing cis- and trans-sides of a microslit filter. In such examples of electronic interrogations, it is understood that the extent of microslit filter occlusion by retained components of interest is directly correlated with any changes in transmembrane electrical resistance or impedance, thus such electronic interrogations can be used to determine the number of components that were retained. Of course, other electronic interrogation methods are possible and these examples have been provided merely for exemplary purposes.

In the various examples of the electronic interrogation methods disclosed herein, the microslit filter-based format may overcome well-known sensitivities of such methods to the presence of electrolytes. For example, most biofluid samples comprise 1 to 200 mM salt concentration. Such salt concentration may deleteriously affect the detection limits of electronic interrogation methods. However, the salt, pH, and/or other electrolyte concentrations may be specified by the one or more filtration and optional washing steps of the methods disclosed herein. For example, salt concentration may be easily altered subsequent to capture and retention of components of interest, by contacting with buffer solutions of specified salt that improves such electronic interrogations.

In various examples, the measurement (e.g., of c)) can further comprise an pressure-dependent modality based on pressure across the microslit filter (e.g., transmembrane pressure). For example, upon retention of desired components by a microslit filter, the openings of such filters are occluded, either partially or fully, such that the fluidic resistance (e.g., resistance against further flow) increases and thus can be measured as an increase in transmembrane pressure. One or more pressure sensors may be used to measure the transmembrane pressure, if placed on opposing cis- and trans-sides of a microslit filter. In such examples, it is understood that the extent of microslit filter occlusion by retained components of interest is directly correlated with any changes in transmembrane pressure, thus such pressure-dependent modalities can be used to determine the number of components that were retained. Of course, other pressure-dependent modalities or methods are possible and these examples have been provided merely for exemplary purposes.

In various examples of the optical imaging and electronic interrogation methods, a signal processing algorithm can be used to record, collect, and compare the optical and electronic data for purposes of detecting the retention of components of interest. Further, the extent of the differences between sample (i.e., used in filtration) and reference (i.e., not used in filtration) microslit filters may be used to quantitate the retention of components of interest within a liquid sample and may require the generation of a series of reference data. Each instance of the series would be recorded, wherein a known number of model components retained by microslit filters are used in each instance of the series. The sample microslit filter data would be compared to the reference series in order to determine the number of components within the liquid sample. A linear regression or other appropriate statistical method may be employed for the comparison of the sample to the reference data. As one example, the signal processing algorithm may compare the intensity or magnitude of fluorescent, absorbance, or electronic data, wherein any differences in the intensity of any such data between reference and sample comprises a potential measure for quantitating the number of components of interest that may be retained by a microslit filter. In this example, it is assumed that retained components that change any such fluorescent, absorbance, or electronic data are dependent on microslit filter occlusion. Thus in some examples, one or more additional reference microslit filters may be used for optical imaging and electronic interrogation methods.

In various examples of the pressure-dependent methods, a signal processing algorithm can be used to record, collect, and compare the pressure data for purposes of detecting the retention of components of interest. Further, the extent of the differences between sample (i.e., used in filtration) and reference (i.e., not used in filtration) microslit filters may be used to quantitate the retention of components of interest within a liquid sample and may require the generation of a series of reference data. Each instance of the series would be recorded, wherein a known number of model components retained by microslit filters are used in each instance of the series. The sample microslit filter data would be compared to the reference series in order to determine the number of components within the liquid sample. A linear regression or other appropriate statistical method may be employed for the comparison of the sample to the reference data. As one example, the signal processing algorithm may compare transmembrane pressure values, wherein any differences in the magnitude of such values between reference and sample comprises a measure for quantitating the number of components

In a further example, the signal processing algorithm may permit the real-time detection of components of interest. For example, quantitative changes in fluorescence, absorbance, or electronic data (when comparing reference and sample microslit filters) may be used in real-time measurements as components are retained by a microslit filter during the filtration step. In this example, any of the flow modalities disclosed herein would be used to capture any components in real-time, and the optical or electronic interrogation method of the present disclosure used to quantitatively detect components as they are retained.

In a further example, the signal processing algorithm may permit the real-time detection of components of interest by a pressure-dependent modality. For example, quantitative changes in transmembrane pressure (when comparing reference and sample microslit filters) may be used in real-time measurements as components are retained by a microslit filter during the filtration step. In this example, any of the flow modalities disclosed herein would be used to capture any components in real-time, and the pressure-dependent methods of the present disclosure used to quantitatively detect components as they are retained.

In other examples, the step of determining the number of retained components can further comprise an optical diffraction modality, wherein such modalities may include, but are not limited to, recording the optical diffraction spectra of microslit filters after the filtration step to observe any captured and retained components of interest.

In an example, a further method for enumerating components of interest from a liquid sample further comprises contacting the liquid sample with a sample microslit filter, such that the components of interest are retained by the microslit filter and undesired components permeate through the microslit filter, optionally, washing the retained components, recording the optical diffraction spectrum of the sample microslit filter (e.g., a sample optical diffraction spectrum), and comparing the sample optical diffraction spectrum to an optical diffraction spectrum of a reference optical diffraction spectrum (e.g., the native microslit filter).

In examples of the optical diffraction method, detecting the retained components is dependent on observable differences between the sample and reference optical diffraction spectra. In its native state (e.g., not used in filtration), the periodic openings of microslit filters can cause coherent light to be diffracted and a repeatedly observable diffraction pattern or spectrum generated. These consistent diffraction spectra can be generated upon trans-illumination with a coherent light source (e.g., laser of specified wavelength) and thus are well-suited to serve as a reference for comparative purposes. However, the retention of components by the sample microslit filter disrupts its periodicity and thus distorts its optical diffraction upon trans-illumination with the same coherent light source. Therefore, observable differences between the sample and reference spectra can be indicative of the presence of the retained components on the sample microslit filter.

In some examples of the optical diffraction method, a signal processing algorithm can be used to record, collect, and compare the sample and reference optical diffraction spectra for purposes of detecting the retention of components of interest. The extent of the differences between sample and reference optical diffraction spectra may be used to quantitate the retention of components of interest within a liquid sample and may require the generation of a series of reference optical diffraction spectra. Optical diffraction spectra from each instance of the series would be recorded, wherein a known number of model components retained by microslit filters used in each instance of the series. The sample optical diffraction spectrum would be compared to the reference series in order to determine the number of components within the liquid sample. A linear regression or other appropriate statistical method may be employed for the comparison of the sample spectrum to the reference series spectra. As one example, the signal processing algorithm may compare the intensity or magnitude of first, second, third, and successive diffraction order peaks, wherein any differences in the intensity of any such diffraction order peaks between reference and sample diffraction spectra comprises a potential measure for quantitating the number of components of interest that may be retained by a microslit filter. In this example, it is assumed that retained components that disrupt the periodicity of trans-illuminated microslit filters may cause quantitative changes in diffraction order peak intensities (e.g., reduction in some peaks, appearance of new diffraction order peaks, or increases in other diffraction order peaks), and that these diffraction order peak changes may be observed when comparing reference and sample diffraction spectra.

In a further example, the signal processing algorithm may permit the real-time detection of components of interest. For example, quantitative changes in diffraction order peaks (when comparing reference and sample diffraction spectra) may be used in real-time measurements as components are retained by a microslit filter during filtration. In this example, any of the flow modalities disclosed herein would be used to capture any components in real-time, and the optical diffraction method of the present disclosure used to quantitatively detect components as they are retained.

In an aspect, a further method for enumerating components of interest from a liquid sample further comprises sorting two or more desired components of interest by selective retention of such two or more populations of components and using one of the measuring methods disclosed herein to quantitate the number of such components. This further method comprises sorting the two or more components of interest into distinct populations based on a physical property of such components (e.g., density, size, or diameter). The sorting of two or more sets of components into distinct populations makes use of two or more specified microslit filters or microslit filter elements.

In a further example, a further method for enumerating components of interest from a liquid sample further comprises:
d) contacting the liquid sample with two or more microslit filters, such that the first population of desired components of interest is retained by a first microslit filter, a second population of desired components of interest is retained by a second microslit filter, and any successive populations of components are retained by successive microslit filters, and any undesired component populations permeate through the microslit filters;
e) optionally, washing the retained components; and
f) measuring the quantity of the two or more populations of retained components to determine their concentration in the liquid sample.

The filtration (e.g., of d)), wherein the two or more components of interest are sorted into distinct populations, may be performed based on the physical properties of each distinct component population (e.g., density, size or diameter, among others) with respect to the filtration properties of the two or more microslit filters. For example, a first microslit filter having rectangular prism openings corresponding in width to the diameter of a first set of components may be used for selective capture and retention of this first component population. A second microslit filter having rectangular prism openings corresponding in width to the diameter of a second set of components may be used for selective capture and retention of this second component population. Any third and successive component populations may be sorted by additional microslit filters with openings corresponding to the diameter of any such third and successive component populations. Of course, microslit filter opening width and component diameter are two of several possibilities and these examples have been provided merely for exemplary purposes.

In examples of the further method, the successive microslit filters may be configured in series (e.g., of different fluidic planes) or in parallel (e.g., of the same fluidic plane), such that the outflows from a preceding microslit filter are in fluidic contact with the inflows of subsequent microslit filters.

For successive microslit filters configured in parallel, the downstream flow of the sample along the parallel fluidic plane allows a first component population to be retained at a first microslit filter, a second component population to be retained at a second microslit filter, and any successive component populations by successive microslit filters, such that the downstream flow of the sample carries components from one microslit filter to successively configured microslit filters.

In various examples of the further method, the liquid sample can bear populations of both biological components and non-biological components. The further methods disclosed herein (e.g., steps d) through f)), in combination with any optional pre-treatment step, can be used to measure the concentration of the one or more populations of biological and non-biological components, and further, the one or more analytical assays described hereunder can be optionally used to determine the composition of such populations of both components.

In examples of the further method, the filtration (e.g., of d)) can be performed using any of the flow modalities previously described for the filtration (e.g., of a)). Any optional washing step (e.g., of e)) can be performed using any of the flow modalities previously described for the optional washing step (e.g., of b)). Further, any of the measuring methods (e.g., of f)) can be performed using any of the modalities previously described for such steps (e.g., of c)). Such measurements can be performed as a single instance or as replicate instances, and reported as a single datum or as mean data.

In an aspect, a further method for enumerating components of interest from a liquid sample further comprises contacting a liquid sample with a microslit filter, wherein the microslit filter comprises a fluidic cavity proximally underlying its filter element, permeating the desired components of interest into this fluidic cavity where they are captured, optionally washing the microslit filter, and measuring the number of captured components within the fluidic cavity by the one or more methods disclosed herein. The openings in the microslit filter are specified for selective permeation of the desired components of interest into the fluidic cavity. Such microslit filter and underlying fluidic cavity structures are disclosed in Striemer et al. (U.S. Pat. Appl. No. 62/248,467), which is hereby incorporated in its entirety by way of reference). It should be noted that for examples related to this aspect of the disclosure, the permeating components are the desired components of interest (unlike other examples previously disclosed herein, where the permeating components were generally undesired components).

In an example, a further method for enumerating components of interest from a liquid sample further comprises:
g) contacting the liquid sample with a microslit filter, such that the components of interest permeate through the openings of the microslit filter and are captured within a fluidic cavity, while undesired components are not captured within said fluidic cavity;
h) optionally, washing the retained components; and
i) measuring the quantity of retained components to determine their concentration in the liquid sample.

The filtration (e.g., of g)), wherein the components of interest selectively permeate through the microslit filter and are captured within fluidic cavities proximally underlying the microslit filter, may be performed based on the physical properties of a component (e.g., density, size or diameter, among others) with respect to the filtration properties of the microslit filters. For example, a microslit filter having rectangular prism openings may be specified such that the width of its openings is larger than the diameter of a component of interest, thus permitting the component to permeate the microslit filter's openings and be captured in its underlying fluidic cavity. Of course, microslit filter opening width and component diameter are two of several possibilities and these examples have been provided merely for exemplary purposes.

In various examples of the further method, the liquid sample can bear populations of both biological components and non-biological components. The further methods disclosed herein (e.g., steps g) through i)), in combination with any optional pre-treatment step, can be used to measure the concentration of the one or more populations of biological and non-biological components, and further, the one or more analytical assays described hereunder can be optionally used to determine the composition of such populations of both components.

The filtration (e.g., of g)), wherein the components of interest selectively permeate through the microslit filter and are captured within fluidic cavities proximally underlying the microslit filter, must consider the range of component sizes within a liquid sample in order to affect the desired filtration outcome. For example, a microslit filter having rectangular prism openings may be specified such that the width of its openings permeates the desired components of interest, but also undesired components that are smaller (e.g., smaller diameter) than that of the desired components of interest. Therefore, the subsequent capture of such a mixture of components would complicate the measurement to determine the number of a particular component of interest (e.g., a mixture problem). Accordingly, as one example to mitigate against this mixture problem), the previously disclosed methods for sorting components into distinct populations (e.g., steps d)-f)) may be used in combination with microslit filters comprising a structure with two microslit filter elements (or two filtration membranes) separated by an intervening fluidic cavity. Such structures with two filtration membranes in one monolithic structure are disclosed in Striemer et al. (U.S. Pat. Appln. No. 62/248,467), which is hereby incorporated in its entirety by way of reference. In one example, a liquid sample is contacted by a microslit filter with a first filtration membrane, the components of interest permeate into a fluidic cavity proximally underlying this first filtration membrane, where such components are captured and retained by a second filtration membrane (e.g., a first distinct component population), and the one or more measurement methods of the present disclosure used to determine the number of components captured in the fluidic cavity that is disposed between the two filtration membranes (e.g., the intervening fluidic cavity). In this example, the rectangular prism openings in the first filtration membrane are specified to permeate the desired components of interest based on a physical property (e.g., diameter), along with other undesired components (e.g., smaller diameter components). The openings in the first filtration membrane are also specified to reject components larger than the size of the first instance of the components of interest. The rectangular prism openings in the second filtration membrane are specified to permeate undesired components based on a physical property (e.g., smaller diameter than the desired components). Successive microslit filters may be used to capture second, third, and any successive distinct component populations by configuring two or more filtration membrane-microslit filter structures in series, such that the outflow of the second filtration membrane of the first microslit filter is in fluidic contact with the first filtration membrane of a second microslit filter, and any successive first membranes of successive microslit filters are in fluid contact with second filtration membranes of subsequently configured microslit filters.

In the various examples, the successively configured microslit filters may be in fluidic connection by a variety of combinations. For example, all of the cis-sides of two or more microslit filters may be in fluid connection, such that the outflow (e.g., sample and those components not retained by the first microslit filter) flow downstream to the inlet of two or more microslit filters. In such an example, the two or more microslit filters would be configured in parallel. In another example, the trans-side of a first microslit filter is in fluidic connection with the cis-side of a microslit filter, such that the outflow of the first microslit filter (e.g., permeate of fractionated sample and any of its components not retained by the first microslit filter) flows to the inlet of the second microslit filter. In this latter example, the two or more microslit filters would be configured in series. Of course, many other configurations are possible and these examples have been merely provided for exemplary purposes.

In examples of the further method, the filtration (e.g., of g)) can be performed using any of the flow modalities previously described for the filtration (e.g., of a)). Any optional washing step (e.g., of h)) can be performed using any of the flow modalities previously described for the optional washing step (e.g., of b)).

In examples of the further method, the measuring methods (e.g., of i)) can comprise optical imaging and/or electronic interrogation methods, as previously described for such steps (e.g., of c)). The measurement methods may further comprise a transmembrane pressure-dependent modality. Such measurements can be performed as a single instance or as replicate instances, and reported as a single datum or as mean data.

In addition to the measurement methods (e.g., of c)) disclosed herein, a first analytical assay may be used to determine the physical characteristics of the retained components. For example, following either c), f) or i), electron microscopy and electron dispersive spectroscopy (EDS) and/or electron energy loss spectroscopy (EELS) may be used to determine additional size and compositional characteristics of the retained components. In some examples, a physical property of a particular component type may be used for its characterization. In addition to the measurement methods (e.g., of c)) disclosed herein, a first analytical assay may be used to determine the physical characteristics of the retained components. For example, following either c), f) or i), electron microscopy and electron dispersive spectroscopy (EDS) and/or electron energy loss spectroscopy (EELS) may be used to determine additional size and compositional characteristics of the retained components. In some examples, a physical property of a particular component type may be used for its characterization. As described above, a fluorescent signal of a cadmium selenide quantum dot, generated at a specified excitation wavelength and recorded at a specified emission wavelength, may be used to confirm that the retained components are cadmium selenide quantum dots. As additional examples of first analytical assays that may be applicable to biological components of interest, following c), f) or i), electron microscopy may be used to characterize the size and/or ultrastructure of the retained components.

As a further example, following either c), f) or i), EDS or EELS may be used to determine additional size and/or composition of the retained components. The elemental and/or molecular compositional data derived from EDS or EELS may provide details on the type of retained component (e.g., the elemental or molecular composition is consistent with a plastic, a polymer, a metal, et cetera). The extent or size of such EDS or EELS spectra may further provide size of the retained component. Such EDS or EELS measurements may be combined with one or more of the optical modalities described herein. For example, optical microscopy may be used to image the size of retained components and EDS or EELS used to gather compositional measurements on the retained components. An algorithm may be used to automate the collection of optical and EDS or EELS measurements. For example, EDS or EELS is used to identify retained components of a certain composition (e.g., polymeric particles), such particles registered (e.g., positionally marked) on the microslit filter, and then optical microscopy used to size the registered and compositionally analyzed/identified particles. In various examples, the EDS or EELS and optical microscopy may be performed one after the other (in any temporal order) or may be performed simultaneously. In one example, the EDS or EELS and optical microscopy methods and related algorithms may be used as a test for polymeric particle (e.g., microplastic) concentration in a sample (e.g., any of the biological or non-biological samples disclosed herein, including a water sample), wherein micro plastic particles are enumerated (e.g., where their concentration is determined) in the sample by any of the other methods of the present disclosure. In another example, the EDS or EELS and optical microscopy methods and related algorithms may be used to evaluate the biological components produced in a cell culture, wherein the concentration of such biological components is determined) in the sample by any of the other methods of the present disclosure, and the EDS or EELS measurements are used to measure the size and/or composition of such biological components. The EDS or EELS and optical microscopy methods may be used for one, two, or more distinct component populations that have been captured and retained by one, two, or more microslit filters.

In a further example, as described above, a fluorescent signal of a cadmium selenide quantum dot, generated at a specified excitation wavelength and recorded at a specified emission wavelength, may be used to confirm that the retained components are cadmium selenide quantum dots. As additional examples of first analytical assays that may be applicable to biological components of interest, following c), f) or i), electron microscopy may be used to characterize the size and/or ultrastructure of the retained components. As described above, the hemoglobin content within retained erythrocytes may be used as the basis of an absorbance optical signal at a specified wavelength for confirming that the captured components are erythrocytes. These first analytical assay methods may be used for one, two, or more distinct component populations that have been captured and retained by one, two, or more microslit filters.

In a further example, a first analytical assay may be Fourier-transformed infrared (FTIR) spectroscopy or Raman spectroscopy that are used to determine the size and/or composition of the retained components. For purposes of this disclosure, FTIR and Raman spectroscopy may be considered an optical modality or method. As an example, following either c), f) or i), FTIR and/or Raman spectroscopy may be used to determine additional size and/or composition of the retained components. The elemental and/or molecular compositional data derived from the FTIR or Raman measurements may provide details on the type of retained component (e.g., the elemental or molecular composition is consistent with a plastic, a polymer, a metal, et cetera). The extent or size of such FTIR or Raman measurements may further provide size of the retained component. Such FTIR and Raman microscopy or spectroscopy measurements may be combined with one or more of the optical modalities described herein. For example, optical microscopy may be used to image the size of retained components and FTIR or Raman microscopy used to gather compositional measurements on the retained components. An algorithm may be used to automate the collection of optical and FTIR or Raman microscopic measurements. For example, FTIR or Raman microscopy is used to identify retained components of a certain composition (e.g., polymeric particles), such particles registered (e.g., positionally marked) on the microslit filter, and then optical microscopy used to size the registered and compositionally analyzed/identified particles. In various examples, the FTIR or Raman microscopy and optical microscopy may be performed one after the other (in any temporal order) or may be performed simultaneously. In one example, the FTIR or Raman and optical microscopy methods and related algorithms may be used as a test for polymeric particle (e.g., microplastic) concentration in a sample (e.g., any of the biological or non-biological samples disclosed herein, including a water sample), wherein micro plastic particles are enumerated (e.g., where their concentration is determined) in the sample by any of the other methods of the present disclosure. In another example, the FTIR or Raman and optical microscopy methods and related algorithms may be used to evaluate the biological components produced in a cell culture, wherein the concentration of such biological components is determined) in the sample by any of the other methods of the present disclosure, and the FTIR or Raman measurements are used to measure the size and/or composition of such biological components. The FTIR or Raman and optical microscopy methods may be used for one, two, or more distinct component populations that have been captured and retained by one, two, or more microslit filters. Of course, other methods for size and compositional analyses may be used and these examples have been provided merely for exemplary purposes.

In various examples, the one or more analytical assays can be performed as either in situ or ex situ measurements, with respect to the fluidic devices used for the contacting and optional washing steps (e.g., steps a) and b), respectively). As an example of an in situ analytical assay, the microslit filter remains within a fully assembled or partially disassembled fluidic device when performing, for example, FTIR or Raman spectroscopy. In this example, the fluidic device may be transferred into an instrument for carrying out an analytical assay. In an example of an ex situ analytical assay, the microslit filter is extracted from a partially or fully disassembled fluidic device and the microslit filter is transferred to an instrument for carrying out, for example, FTIR or Raman spectroscopy or electron microscopy, EDS, or EELS. In this latter example, the extracted microslit filter may be optionally transferred to another substrate prior to transfer into the instrument carrying out an analytical assay. In another example, the step of enumerating the retained components (e.g., either step c), f) or i)) is performed as an in situ measurement that is then followed by an ex situ analytical assay.

In a further example of first analytical assays, a retained component may be labeled with a unique binding agent, wherein each unique binding agent bears an unique enzyme, chromophore, fluorophore or quantum dot that endows a distinct optical signal to each retained component, upon reaction with the appropriate reagents (in the case of enzymes) or upon illumination at the appropriate wavelength (in the case of chromophores, fluorophores, and quantum dots). These binding agents may be conjugated to an optical detection moiety, wherein these optical detection moieties may include, but are not limited to, a fluorophore, a chromophore, a fluorescent polymeric nanocomponent, a quantum dot, or an enzyme or other catalytic molecule which exhibits or participates in substrate reduction process (or processes), such that these conjugates possess or yield an emission, a chemiluminescent or absorbance signal at a defined wavelength or range thereof. Further, substrates for enzymatic or catalytic reduction, as well as any required co-reagents for such reduction, may be added sequentially or may be concurrently added with binding agents. These first analytical assay methods may be used for one, two, or more distinct component populations that have been captured and retained by one, two, or more microslit filters. Of course, other first analytical assay methods may be used and these examples have been provided merely for exemplary purposes.

For purposes of this disclosure, a binding agent (e.g., affinity moiety) possesses specific molecular binding capacity, with a relatively high association rate and low disassociation rate for its cognate target binding molecule or ligand (e.g., retained component of interest). It is generally recognized that for practical purposes, the binding agent's relatively high association rate and low disassociation rate for its ligand should result in the binding agent possessing an equilibrium disassociation constant (Kd) that are within the range of pM to nM values. In examples wherein the binding agent is a biomolecule, the three-dimensional structure of the binding agent is such that it can form high-affinity interactions upon binding of its ligand through, for example, electrostatic, hydrophobic, ionic, van der Waal, and/or hydrogen-bonding interactions, among others. For example, the three-dimensional structure of monoclonal, polyclonal, or fragment of an antibody is determined by the amino acid sequence of these proteins, and more particularly, the specific and unique amino acid sequences of the Fv or FaB regions of such proteins determines its affinity for the epitopes of a ligand. As another example, the three-dimensional structure of lectins, and in particular, the specific and unique amino acid sequence of its carbohydrate-binding region determines its affinity for carbohydrate structures of its ligands. As an additional example, the three-dimensional structure of an aptamer is determined by its nucleic acid sequence, such that the resulting three-dimensional structure of the aptamer forms high-affinity binding interactions sites with regions of its ligands. As another example, the nucleic acid sequence of an oligonucleotide determines its sequence-specific binding to complementary nucleic acid sequences through canonical base-pairing interactions. Exemplary binding agents can be chosen from among classes of affinity moieties that include, but are not limited to, monoclonal or polyclonal antibodies or fragments derived from such antibodies, DNA or RNA oligonucleotides or aptamers, peptides or modified peptide derivatives, lectins, bacteriophages, small molecules, proteins or their domains with known protein or nucleic acid binding capacity, among others. Of course, many other possible biomolecular structural interactions with target ligands are possible and the examples have been provided merely for exemplary purposes. In the various examples disclosed herein, these exemplary interactions (as well as other possible interactions) may describe the manner in which binding agents interact with retained components of interest for purposes of carrying out a first analytical assay.

For purposes of this disclosure, a ligand represents a portion of a retained component of interest with which a binding agent interacts. For example, a ligand may be the epitope of an analyte bound by a monoclonal antibody or the epitopes of an analyte bound by a polyclonal antibody.

As a further method, an optional elution step may follow c), f) or i), for purposes of carrying out a second analytical assay on eluted components. For example, following c), f) or i), retained components can be transferred to another appropriate receptacle (e.g., vessel, surface, instrument, or the like) for subsequent second analytical assays. Elution of retained components may be performed by introducing a bolus of buffer solution to flush any retained components from the microslit filter by reversing any of the flow modalities used for initial retention of such complexes (e.g., during a)). Second analytical assays include, but are not limited to, assays well-known to those skilled in the related art, and may include, but are not limited to, a sequencing reaction, an amplification reaction, polymerase chain reaction, reverse transcriptase-polymerase chain reaction, ligase chain reaction, Northern blotting, Southern blotting, fluorescent hybridization, enzymatic treatment, labeling with second binding agents, enzyme-linked immunosorbent assay, immunocytochemistry, Western blotting, immunoprecipitation, fluorescence-activated sorting, optical imaging, electron microscopy, surface plasmon resonance, Raman spectroscopy, mass spectroscopy, Fourier-transformed infrared spectroscopy, or interferometry, among other possibilities. In some examples, the assay can be nanopore-based resistive pulse sensing; for example, as disclosed in Huff et al. (WO2016161402A1). In other examples, the assay can be arrayed imaging reflectometry (for example, as disclosed in Miller and Rothberg, U.S. Pat. No. 7,292,349). If multiple components of interest have been retained, then assays for multiplex detection may be used to analyze these two or more components. Of course, other possibilities exist and these examples have been provided merely for exemplary purposes.

In an example of the methods disclosed herein, the liquid sample is whole blood and three microslit filters (as independent or combined configuration) are specified such that the first microslit filter captures and retains leukocytes, the second microslit filter captures and retains platelets, and the third microslit filter captures and retains erythrocytes. In other examples, various combinations of the three microslit filters captures and retains these distinct cell types from whole blood. These examples wherein the three major cell types from whole blood are enumerated (e.g., where their concentration is determined) is well-known to those skilled in the related art and is commonly referred to as a complete blood cell count (i.e., CBC). The whole blood may be diluted prior to contact with the various microslit filters, and such dilution accounted when algorithms of the present disclosure calculate the number of cells per volume of blood passed through a fluidic device. Each cell type may be measured as a single instance or as replicate instances, as the number of cells reported as a single datum or mean data. Additionally, the first analytical assay methods disclosed herein may be used to differentiate retained cell types following their enumeration. For example, major subtypes of leukocytes (e.g., neutrophils, eosinophils, basophils, lymphocytes, monocytes, and the like) may be identified using binding agents such as, for example, antibodies with an optical detection moiety as previously disclosed herein. Each antibody would be directed to bind a particular leukocyte subtype, thus permitting the differentiation of captured leukocytes into their major subtypes, Further, similar algorithms may be used to enumerate these major subtypes. As another example, other binding agents may be used to differentiate blood group antigens (e.g., A, B, O and/or Rh) on erythrocytes. These differentiated analyses are well-known to those skilled in the related art and are commonly referred to as complete blood cell counts (CBC) with differentiation. The whole blood sample may be obtained from the donor by means of a finger prick, a venous puncture blood draw or an arterial blood draw.

In some examples of the methods disclosed herein, wherein the retained component of interest is a living organism (e.g., among those listed above: cells, bacteria, fungi, or the like), an additional incubation period providing time for culture, growth and/or expansion of the isolated living species may be incorporated. In these examples, the living cells are enumerated by the measuring methods (e.g., of c) and then may be eluted by an optional elution step for second analytical assays or other subsequent analyses. first or Second analytical assays may be those disclosed herein, while other subsequent analyses may include, but are not limited to, phenotypic or functional tests, following or during the additional incubation period. Of course, the phenotypic or functional assays may be combined with one or more of the previously listed first or second assays. During the additional incubation period, appropriate environmental conditions and nutrient supplies are furnished to the living species, having eluted them from the isolating microslit filters and transferred them to vessels appropriate for their culture. The sterility of the collected biofluid sample, the device and reagent components, and the related methods should be maintained for proper growth and culture. As an example of the additional incubation period and phenotypic/functional assay, an isolated circulating tumor cell may be clonally expanded and the growth of multiple derived clonal cultures challenged by a panel of chemotherapeutic agents to identify potential best therapies. As another example, an isolated bacterium may be clonally expanded and the growth of multiple derived clonal cultures challenged by a panel of antibiotic agents to assess phenotypic antibiotic resistance.

In an example of the methods disclosed herein, the liquid sample is any one of the sample types disclosed herein and three microslit filters (as independent or combined configuration) are specified such that the first microslit filter captures and retains large polymeric particles (e.g., ≥20 μm diameter microplastic particles), the second microslit filter captures and retains medium polymeric particles (e.g., 10 μm to 20 μm diameter microplastic particles), and the third microslit filter captures and retains small polymeric particles (e.g., ≤10 μm diameter microplastic particles). In other examples, various combinations of microslit filters captures and retains distinct sets of polymeric particles from a sample. Of course, any combination of microslit filter opening sizes may be used in order to accomplish the desired capture and retention of particular populations of polymeric particles. These examples wherein sets of particle populations from a sample are enumerated (e.g., where their concentration is determined) may be useful as a test for microplastic particle contents in the sample and thus may be referred to as “a microplastic particle test” throughout the present disclosure. As examples, a microplastic particle test may be useful for determining the presence or absence of such polymeric particles in water samples drawn, for example, from a tap in a home or other building, in the effluent from a industrial process or a water sanitation process, from a water treatment process, among other possibilities. Similarly, such methods may be used to enumerate polymeric particle content with a food sample (e.g., milk, juice, beer, wine, among other possibilities). Any of these samples may be diluted prior to contact with the various microslit filters, and such dilution accounted when algorithms of the present disclosure calculate the particle number per volume passed through a fluidic device. The foregoing examples may be combined with one or more analytical assays (e.g., FTIR or Raman spectroscopy, electron microscopy, optical microscopy, among others) to determine additional size and compositional measurements on the polymeric particles. Each particle type may be measured as a single instance or as replicate instances, and the number of particles reported as a single datum or mean data.

In another example of the methods disclosed herein, the liquid sample is conditioned cell culture media or other fluids derived from cell culture (e.g., a cell lysate) that bear the biological components of interest (e.g., a protein, a virus, or an extracellular vesicle, among others), and three microslit filters (as independent or combined configuration) are specified such that the first microslit filter captures and retains large components (e.g., ≥8 μm diameter), the second microslit filter captures and retains medium components (e.g., <8 μm to >2 μm diameter), and the third microslit filter captures and retains small components (e.g., <2 μm and >0.5 μm diameter). In other examples, various combinations of microslit filters captures and retains distinct sets of biological components from a cell culture-derived liquid sample. Of course, any combination of microslit filter opening sizes may be used in order to accomplish the desired capture and retention of particular populations of biological components. These examples wherein sets of biological component populations from a sample are enumerated (e.g., where their concentration is determined) may be useful as a test for evaluating the production of such biological components in a cell culture (e.g., a cell, a protein, a virus, or an extracellular vesicle). For example, such a test may be used as quality control and/or process control measurements during the production of biological components in a cell culture. Accordingly, this test may be referred to as “a biological component production test” throughout the present disclosure. For example, the culture may be a suspension culture of genetically modified Chinese hamster ovary cells secreting humanized monoclonal antibody. In another example, the culture may be a suspension culture of human embryonic kidney cells genetically modified to produce a lenti virus vector. In another example, the culture may be a suspension culture of genetically modified T-cells for adoptive cell transfer therapy (e.g., chimeric antigen receptor T-cell therapy). In another example, the culture may be an adherent culture of autonomous mesenchymal stem cells secreting extracellular vesicles for therapeutic applications. A biological component production test may be used at various production-related steps throughout the production of such biological components to evaluate quality and process controls. The samples for such tests may be drawn prior, during, or after completion of any production-related steps and thus the tests may be recognized as “in-line” or “at-line” tests by those skilled in the related art. Those skilled in the related art will also recognize that these production-related steps may include, but are not limited to, incubation, expansion, clarification, chromatography, sterile filtration, buffer exchange, and the like, and the tests of the present disclosure used to optimize and/or characterize these production-related steps. In one preferred example, a biological component production test of the present disclosure is used to evaluate production-related steps and conditions that either promote or reduce aggregates of the biological components of interest, as well as distinguish the various aggregation states (e.g. monomer, dimer, trimer, etc.) of the biological component of interest. Any of these samples may be diluted prior to contact with the various microslit filters, and such dilution accounted when algorithms of the present disclosure calculate the component number per volume passed through a fluidic device. The foregoing examples may be combined with one or more analytical assays (e.g., FTIR or Raman spectroscopy, electron microscopy, optical microscopy, among others) to determine additional size and compositional measurements on the biological components. Each biological component may be measured as a single instance or as replicate instances, and the number of components reported as a single datum or mean data.

In various examples, the methods of the present disclosure may comprise using a filter comprising one or more or a plurality of microslits and/or one or more or a plurality of nanopores. The method may further comprise enumerating one or more components retained on the filter comprising one or more or a plurality of microslits and/or one or more or a plurality of nanopores.

In various examples, a method comprises contacting a liquid sample with a mircoslit filter (e.g., filtering with a microslit filter), followed by contacting the liquid sample with a nanoporous filter. The method may further comprise enumerating one or more components retained on the microslit filter and/or nanoporous filter.

In an aspect, the present disclosure also provides fluidic devices for carrying out the methods of the disclosure. In various examples, these fluidic devices can include, but are not limited to, one or more microslit filters for capturing and retaining components of interest from a liquid sample. In an example, the fluidic device comprises at least one microslit filter. In another example, the fluidic device comprises two or more independent microslit filters. In another example, the fluidic device comprises at least one microslit filter with two or more microslit filter elements, wherein the microslit filter element comprises microslit filter aspects within one substrate.

In various examples, a device of the present disclosure may comprise one or more microslit filter(s), each comprising a plurality of microslits, and one or more nanoporous filter(s), each comprising a plurality of nanopores. In various other examples, a device of the present disclosure may comprise a filter having one or more or a plurality of microslits and/or one or more or a plurality of nanopores. Such devices are suitable for use in methods of the present disclosure.

In an example, the present disclosure provides a fluidic device comprising at least one microslit filter with a membrane thickness of ≤500 nm and membrane apertures (e.g., openings) with an aspect ratio of ≥1:100, at least one light source, and at least one detector. In another example, the present disclosure provides a fluidic device comprising at least one microslit filter with a membrane thickness of ≤500 nm and membrane apertures with an aspect ratio of ≥1:100, and at least one transmembrane pressure sensor.

In a further aspect, the thickness (e.g., membrane thickness), porosity, and opening aspect ratio of microslit filters are specified in examples disclosed herein, such that the thickness and porosity properties promote low species permeation resistance and high permeability (e.g., high permeation capacity, low pressure operation), while the opening aspect ratio promotes non-fouling behavior and precision molecular cut-off (e.g., selective retention of components and selective permeation of other components). In further examples, the characteristics of microslit filters are specified for performing an upstream sample preparation. In various examples, the fluidic devices may be constructed such that the incorporated microslit filters can be optionally extracted from the fluidic device (upon partial or complete disassembly of the fluidic device) for carrying out the various methods of the present disclosure.

In an example of the present disclosure, a device for capturing and retaining a component of interest from a liquid sample comprises a fluidic device comprising one or more microslit filters or microslit filter elements, having at least one chamber or channel in fluidic contact with the cis-side of the microslit filter and at least one chamber or channel in fluidic contact with the trans-side of the microslit filter, wherein the cis and trans-sides oppose each other, and the cis- and trans-side chambers or channels are fluidically connected by a plurality of openings in the one or more microslit filters.

The microslit filters of the fluidic device have specified physical characteristics to promote high cis-to-trans permeation of liquid samples and to promote non-fouling behavior and precision molecular cut-off. The microslit filters comprise a plurality of arrayed openings that fluidically connect/communicate with their opposing cis- and trans-sides. The microslit filter can have a range of membrane thickness; for example, the membrane thickness can be from 50 nm to 25 μm, including all values to the nm or μm and ranges therebetween. In another example, the membrane thickness can be from 50 nm to 1 μm, including all values to the nm or μm and ranges therebetween. In another example, the membrane thickness can be from 50 nm to 500 nm, including all values to the nm and ranges therebetween. The microslit filters can have a range of porosity; for example, the porosity can be from less than 1% to 75%, including all integer % values and ranges therebetween. In another example, the porosity can be from 5% to 30%, including all integer % values and ranges therebetween. The microslit filter can have a range of aspect ratio for its openings; for example, its openings can be cubic prisms, rectangular prisms, or trapezoids and be 0.5 μm to 15 μm in width and 5 μm to 100 μm in length, including all values to the μm and ranges therebetween thus possessing a range of aspect ratio (in terms of width to length) between 1:0.33 to 1:200. In a particular example, the microslit filter is 400 nm thick, has approximately 17% porosity, and has openings of 9 μm width and 50 μm length, and an aspect ratio of 1:5.5. In another particular example, the microslit filter is 400 nm thick, has approximately 9% porosity, and has openings of 1 μm width and 50 μm length, and an aspect ratio of 1:50. Of course, other values are possible and these are merely listed as examples. In another particular example, the microslit filter (e.g., the membrane) is 400 nm thick, has approximately 11% porosity, and has openings of 0.5 μm width and 50 μm length, and an aspect ratio of 1:100. In another particular example, the microslit filter (e.g., the membrane) is 200 nm thick, has approximately 30% porosity, and has openings of 0.2 μm width and 10 μm length, and an aspect ratio of 1:50.

The microslit filters of the fluidic devices should be further specified so that contacting the liquid sample performs the desired filtration steps of the present disclosure. Accordingly, the microslit filters of the fluidic device should be specified in terms of the width of their openings for selective capture and retention of components and for selective permeation of other components. Further, the relative size of components to be retained in an upstream sample preparation should be considered with respect to the opening width of the microslit filter used for such purposes.

The fluidic device may accomplish the filtration (e.g., of a)) using several flow modalities and filtration system configurations. In an example, the fluidic device uses dead-end filtration, wherein contact with the microslit filter involves flow that is normal to the microslit filter surface. The liquid sample is introduced to the cis-side of the filter and either hydrostatic pressure or the application of cis-side positive pressure or trans-side negative pressure initiates flux from the cis- to the trans-side of the filter. Positive and negative pressures may be generated by, for example, gravity/hydrostatic pressure or by pumping, vacuum, gas pressurization, centripetal force and the like. In various examples, the fluidic device is a stirred cell dead-end filtration system that uses gas pressurization or vacuum, or the fluidic device is a centrifuge insert dead-end filtration system that uses centripetal force. In another example, the fluidic device uses transmembrane pressure differential and tangential flow, wherein contact with the microslit filter involves flow that is tangential to the microslit filter surface, the biofluid or the liquid sample is introduced to the cis-side of the filter, bulk flow is initiated on both cis- and trans-sides of the filter such that a transmembrane pressure is generated (e.g., relative negative pressure on the trans-filter side), thus initiating flux from the cis- to the trans-side of the filter. The relative bulk flow rate on the cis-filter side should be greater than the bulk flow rate on the trans-filter side in order to create the desired transmembrane pressure vector. These tangential flow and transmembrane flow vectors create diafiltration as described previously herein. In various examples, the cis- and trans-sides of the microslit filters are fluidically connected to chambers or channels of these opposing sides and bulk flow is initiated in these chambers or channels using gas pressurization or pumping apparatus (e.g., a syringe or peristaltic pump, or the like). In various examples, the fluidic device is a tangential flow filtration system.

The fluidic device of the present disclosure can further comprise two or more independent microslit filters in a variety of configurations. In one example, a liquid sample is passed from the cis-side of a first microslit filter to its trans-side, and then from the trans-side of a first microslit filter to the cis-side of a second microslit filter, and then to the trans-side of a second microslit filter. In another example, a liquid sample is passed from the cis-side of a first microslit filter to the cis-side of a second microslit filter. As another example, a liquid sample is passed from the cis-side of a first filtration membrane of a first microslit filter to the trans-side of a first filtration membrane and into a first intervening fluidic cavity, then is passed from the first intervening fluidic cavity to the cis-side of a second filtration membrane of a first microslit filter and then to the trans-side of the second filtration membrane. Then, the liquid sample is passed from the cis-side of a first filtration membrane of a second microslit filter to the trans-side of a first filtration membrane and into a second intervening fluidic cavity, then is passed from the second intervening fluidic cavity to the cis-side of a second filtration membrane of a second microslit filter to the trans-side of the second filtration membrane of the second microslit filter, and so on. Thus, the two or more independent microslit filters can be disposed in different fluidic planes (e.g., in series) or disposed on the same fluidic plane (e.g., in parallel). In various examples, the independent microslit filters can be connected by tubing or channels with inlets and outlets between the two or more microslit filters. As an example, a fluidic device incorporating at least two independent microslit filters may be used to perform an upstream sample preparation and component enumeration method of the present disclosure. As another example a fluidic device incorporating at least three independent microslit filters may be used to perform a method of the present disclosure (e.g., a complete blood cell count, a microplastic test, or a biological component production test). Of course, other fluidic device configurations are possible and these examples have been provided merely for exemplary purposes.

In another example, the fluidic devices further comprise one or more microslit filters with two or more filtration membranes (e.g., microslit filter elements). In one example, the fluidic device comprises a microslit filter with multiple filtration membranes disposed on a combined substrate. As one example using a microslit filter with multiple filtration membranes in a combined substrate configuration, a liquid sample is initially contacted by a first microslit filtration membrane, the fluid is then passed to and contacted by a second filtration membrane, and then the fluid is passed to and contacted by any third and/or successive filtration membranes. In this example, the successive and multiple filtration membranes (e.g., microslit filter elements) are within one combined substrate and are disposed along the length of one channel, such that the fluid is successively contacted by each element as it passes through the channel and thus the elements are all of the same fluidic plane. Such substrates are fabricated with the various microslit filter elements (e.g., filtration membranes) on distinct regions of one substrate. As one example, a fluidic device incorporating a combined microslit filter with at least three filtration membranes (or elements) may be used to perform a complete blood cell count method of the present disclosure. Of course, other fluidic device configurations are possible and these examples have been provided merely for exemplary purposes.

The fluidic devices may accomplish any optional washing steps (following the filtration steps) using similar flow modalities and filtration system configurations as those used for filtration steps. For example, in a stirred cell filtration system or a centrifuge tube insert filtration system (both of which are configured for dead-end/normal flow), one or more bolus of fresh buffer may be introduced to the cis-side of the microslit filter (following the filtration step), and either hydrostatic pressure or the application of cis-side positive pressure or trans-side negative pressure initiates flux of the buffer. In an additional example, in a tangential flow filtration system (configured for tangential flow), one or more bolus of fresh buffer may be introduced to the cis-side of the microslit filter (following the filtration steps), and bulk flow on the cis-side used to initiate buffer flow. Application of transmembrane pressure would be optional in this latter example.

The fluidic devices may accomplish any optional elution steps using similar flow modalities and filtration system configurations as those used for filtration steps. However, in some examples, the fluidic devices should be able to operate the flow modality in reverse of that initially used for the filtration steps. As one example, the retained components may be eluted from the cis-side of a microslit filter by introduction of a bolus of buffer to the cis-side of the contacting microslit filter, resuspension of the components by any mechanical mixing method, and the re-suspended components removed from the fluidic device (e.g., by a manual or automated pipet). As another example, the pressurization within a stirred cell filtration system may be reversed, following an introduction of fresh buffer to the cis-side of the contacting microslit filter, and the pressurization used to flush off retained components into the buffer. As yet another example, the relative bulk flow rates and direction of the transmembrane pressure vector may be reversed in a tangential flow system to flush off retained components into a flow of fresh buffer introduce to the cis-side of the contacting microslit filter.

The various configurations of fluidic devices comprising two or more microslit filters may use tangential flow alone or tangential flow in combination with normal flow. As an example, tangential flow alone can be used to contact a liquid sample by the cis-sides of successively configured microslit filters in either the independent or combined configurations if these elements are all of the same plane. As a different example, tangential flow can be used to contact a liquid sample by the cis-sides of two successively configured microslit filters (these two elements being of the same plane), and then normal flow used to pass the resultant permeate of the second microslit filter to the cis-side of a third microslit filter (the second and third microslit filters being of different planes). The fluid can be successively transferred between elements either as continuous flow (passing fluid through all elements in uninterrupted succession) or as discrete flow (passing fluid one element at a time with interrupted flow at each element). Tangential and normal flow within these exemplary fluidic devices would be initiated by the same bulk flow modalities described above. Accordingly, the further examples of the fluidic device can be a tangential flow filtration system or a combined tangential/normal flow filtration system.

The microslit filters of the fluidic devices comprise porous materials that can be fabricated by various methods. For example, these filters and membranes may comprise a suspended membrane layer that was fabricated by: 1) patterning and etching the openings into silicon nitride or other Si-based film, using well-known photolithography and reactive ion etching methods, followed by etching to suspend the silicon nitride or other Si-based film by etching through a substrate (e.g., a silicon wafer); 2) embossing openings into polyurethane or poly-dimethyl-siloxane, using a master mould with a negative relief pattern of the openings; 3) solvent-casted polymer membranes of appropriate thickness and opening size and aspect ratio; 4) track-etched polymer membranes of appropriate thickness and opening size and aspect ratio; 5) patterned photoresist membranes (e.g., SU-8) with openings of appropriate size and aspect ratio that are fabricated by well-known photolithography methods (e.g., patterning SU-8 photoresist on a Si wafer and subsequent lift-off of the SU-8 membrane after its patterning); or 6) stainless steel, nickel or other alloy membrane with electro-formed openings of appropriate size and aspect ratio. In a further example, the microslit filter, with at least one first filtration membrane, at least one intervening fluidic cavity, and at least one second filtration membrane, is fabricated by methods disclosed in Striemer et al. ((U.S. Pat. No. 62/248,467), which is hereby incorporated in its entirety by way of reference), wherein microslit filters may be fabricated by transmembrane etching through their openings (e.g., as openings in a silicon nitride layer disposed onto a Si wafer substrate). Of course, other microslit filter materials and fabrication methods are possible and these examples have been provided merely for exemplary purposes.

The microslit filters of the fluidic devices have a specified thickness so that the fluidic device operates at a range of low pressurization when performing the filtration steps; e.g., 10 Pa to 1.0 kPa, and all Pa values therebetween.

Microslit filters are incorporated into fluidic devices as independent or combined elements for carrying out the methods of the disclosure. For example, one or more microslit filters, each comprising a suspended silicon nitride membrane on a silicon substrate, can be fabricated using a Si wafer and the resultant microslit filter(s) incorporated into a fluidic device. As another example, two or more aspects of a Si wafer can be fabricated to yield two or more microslit filter elements (e.g., aspects) and the resultant microslit filter with two or more elements incorporated into a fluidic device. The suspended silicon nitride membranes correspond to freestanding regions wherein the plurality of openings through the membranes (e.g., cubic or rectangular prisms) fluidically connect cis- and trans-sides of the membranes.

Although the various examples disclosed herein use microslit filters in preferred embodiments and examples, other types of silicon-based membranes or filters could be used in the methods of the present disclosure. For example, the membrane could be a nanoporous silicon nitride membrane (NPN). Examples of NPN membranes and the fabrication of such membranes are disclosed in U.S. Pat. No. 9,789,239 (Striemer et al. “Nanoporous Silicon Nitride Membranes, and Methods for Making and Using Such Membranes”), the disclosure of which with regard to NPN membranes is incorporated herein by reference. In another example the membrane could be a microporous silicon nitride membrane (MP SiN). Examples of MP SiN membranes and the fabrication of such membranes are known in the related art. In yet another example, the membrane could be a microporous flat tensile silicon oxide membrane (MP SiO₂). Examples of MP SiO₂ membranes and the fabrication of such membranes are disclosed in U.S. Pat. No. 9,945,030 (Striemer et al. “Free-Standing Silicon Oxide Membranes, and Methods of Making and Using Same”), the disclosure of which with regard to MP SiO₂ membranes is incorporated herein by reference.

The microslit filters can be functionalized with moieties that decrease the adhesion to liquid sample constituents (e.g., coatings to reduce or prevent fouling). For example, a silanization process may be used for functionalizing microslit filters, wherein a vapor phase silane source contacts the filters or membranes, the silane reacts with a functional surface group of the filters or membranes, and the silane is further derivitized with a poly-ethylene glycol moiety of 5-10 carbon atoms in length. As another example, the microslit filters may be functionalized with a carbinylation process as disclosed in Shestopalov et al. ((U.S. Pat. No. 9,089,819), the disclosure of which is hereby incorporated in its entirety by way of reference), wherein a vapor phase carbine source (e.g., a diazirine compound) contacts the filters, the carbine reacts with a functional surface group of the filters or membranes (e.g., an aliphatic monolayer), and the carbine is further derivitized with a poly-ethylene glycol moiety of 5-10 carbon atoms in length. In a further example, the microslit filter is functionalized using a epihalohydrin-based process as disclosed in Carter and Roussie ((U.S. Provisional Pat. Appln. No. 62/614,232), the disclosure of which is hereby incorporated in its entirety by way of reference), wherein silicon nitride microslit filters are chemically treated, reacted with gas-phase epichlorohydrin, and then terminally reacted with an amine-containing reactant (e.g., amino-PEG or ethanolamine). In these examples, the terminal surface hydroxyl groups provide a non-fouling surface (e.g., a non-fouling surface treatment). Furthermore, such hydroxylated surfaces may improve eluting of retained components, if their elution is desired. Alternatively, these functionalization processes may be used for functionalizing the surfaces of microslit filters to promote their selective interactions with certain components. For example, a silane, carbene, or epihalohydrin-treated microslit filter may be further functionalized with one or more binding agents disclosed herein to promote capture and retention of components bearing target ligands for such binding agents. As another example, the capture and retention of negatively charged components at neutral pH (e.g., silica) may be improved if microslit filters where functionalized with terminal moieties that endowed them with positive charge at neutral pH (e.g., a moiety with a primary amine of pKa in the range of 7-8). As another example, surface functionalization may be used to promote the permeation of undesired components likely to be present in a liquid sample (e.g., by repelling the undesired components by a charged surface of similar polarity). Of course, other possible surface functionalization possibilities exist and these examples have been provided merely for exemplary purposes.

The fluidic device may further comprise a light source and a detector for recording optical signals of any first and/or second analytical assay. The light source may be a laser, a photodiode array, or any similar light source. The detector may be a spectrophotometer, a photometer, a leumeter, a charge-coupled device, or any other similar device. The light source and detector may be appropriate for carrying out the optical imaging, optical diffraction, and optical-dependent second analytical assays or modalities, either for determining their number or other characterization or compositional analyses. For example, the light source and the detector would be configured as required by the optical imaging or optical diffraction methods and used to read optical signals generated at the microslit filters; for instance, the microslit filter or sorting membrane is disposed between the light source and the detector.

The fluidic device may further comprise an algorithm for calculating the concentration of components in a liquid sample, from a known or measured volume of liquid sample passed in the fluidic device and from the determined number of components of interest captured and retained on the microslit filter, and may further account for any dilution of the liquid sample (if applicable). The fluidic device may further comprise a flow meter for measuring the volume of liquid sample passed in the fluidic device. In an example, the fluidic device comprises an algorithm for calculating component concentration. In another example, the fluidic device comprises a flow meter for measuring volume passed of the liquid sample.

In an example, a fluidic device for optical imaging and/or optical diffraction modalities for determining the number of retained components of interest comprises at least one sample microslit filter, at least one reference microslit filter, a light source, and a detector, and can carry out the methods of the optical imaging and optical diffraction modalities, as well as optical-dependent second analytical assays. In some examples, the sample and reference microslit filters are disposed between the light source and the detector, such that the microslit filters can be trans-illuminated by the light sources and their resultant imaging or diffraction spectra recorded by the detector. In a further example, a fluidic device for such optical modalities may further comprise an additional microslit filter, such that the additional microslit filter performs sample preparation prior to the optical modality methods. Such fluidic devices may further comprise a signal processing algorithm, wherein the signal processing algorithm automates the collection and comparison of sample and reference microslit filter imaging and/or diffraction spectra. In some examples, multiple fluidic devices of the optical modalities may be configured such that multiple components of interest (from one liquid sample) can be analyzed on multiple sample microslit filters in parallel.

The fluidic device may further comprise one or more pairs of electrodes, a voltmeter, and/or a function generator for carrying out an electronic interrogation modality based on transmembrane electrical resistance or impedimetric spectroscopy.

In an example, a fluidic device for electronic interrogations for determining the number of retained components of interest comprises at least one sample microslit filter, at least one reference microslit filter, a voltmeter, and at least one pair of electrodes. In other examples, the fluidic device may further comprise a function generator. Such fluidic devices can carry out the electronic interrogation methods of the present disclosure. In some examples, the sample and reference microslit filters are disposed between the one or more pairs of electrodes, such that the transmembrane electrical resistance and/or impedance recorded by a detector. In a further example, a fluidic device for electronic interrogations may further comprise an additional microslit filter, such that the additional microslit filter performs sample preparation prior to the electronic interrogations. Such fluidic devices may further comprise a signal processing algorithm, wherein the signal processing algorithm automates the collection and comparison of sample and reference microslit filter transmembrane electrical resistance and/or impedance. In some examples, multiple fluidic devices of the electronic interrogation may be configured such that multiple components of interest (from one liquid sample) can be analyzed on multiple sample microslit filters in parallel.

In an example, a fluidic device for pressure-dependent modalities for determining the number of retained components of interest comprises at least one sample microslit filter, at least one reference microslit filter and one or more pressure sensors (e.g., transmembrane pressure sensor), and can carry out the methods of pressure-dependent modalities. In some examples, the sample and reference microslit filters are disposed between the one or more pressure sensors, such that the microslit filters' transmembrane pressure (e.g., fluidic resistance) can be measured. In a further example, a fluidic device for such pressure-dependent modalities may further comprise an additional microslit filter, such that the additional microslit filter performs sample preparation prior to the transmembrane pressure measurements. These fluidic devices may further comprise a signal processing algorithm, wherein the signal processing algorithm automates the collection and comparison of sample and reference microslit filter transmembrane pressure. In some examples, multiple fluidic devices of the pressure-dependent modalities may be configured such that multiple components of interest (from one liquid sample).

In an aspect, the present disclosure provides a kit comprising one or more specified devices and, optionally, one or more reagents for carrying out the methods of the disclosure, wherein the physical properties of microslit filters (e.g., thickness, porosity and opening aspect ratio and size) are specified relative to the size of components of interest. Accordingly, the devices and reagents of the disclosed kit are intended to be used as a combined system.

In an example, a kit for capturing and retaining components of interest from a liquid sample comprises one or more fluidic devices, wherein the fluidic device of the kit comprises one or more microslit filters or more microslit filters with one or more filter elements. The width of the microslit filters' openings are specified relative to the size of the components of interest Cath are to be captured and retained by the one or more microslit filters (e.g., the components' diameter is greater than the width of the microslit filters' openings). The kit comprises elements to perform one or more method of the present disclosure.

In an example, a kit of the present disclosure further comprises one or more pump, one or more flow meter, one or more centrifuge, and one or more algorithm for component concentration calculation, for carrying out any of the flow modalities of the present disclosure.

In an example, a kit of the present disclosure further comprises one or more algorithm for component size and/or composition determinations.

In an example, the kit of the present disclosure may further comprise one or more pairs of electrodes, one or more voltmeter, and one or more function generators, for carrying out the electronic interrogation methods of the present disclosure.

In an example, the kit of the present disclosure may further comprise one or more pressure sensors (e.g., transmembrane pressure sensor) for carrying out the transmembrane pressure measurements of the present disclosure.

In a further example, a further kit comprises one or more buffer, one or more wash or elution solution, one or more binding agent, a light source, and a detector. The one or more binding agents comprise affinity moieties that bind ligands of the retained components on microslit filters. The light source and the detector of the kit comprise elements for carrying out first and/or second analytical assays on any retained components retained on microslit filters. The one or more buffer, washing, and/or elution solutions comprise reagent solutions for carrying out any optional washing or elution steps. In further examples, a further kit of the present disclosure comprises plasmonically active microslit filters and thermal elements to specify temperature during optical imaging.

In a further example, a further kit comprises one or more additional microslit filters for performing upstream sample preparation.

The fluidic device of the optical diffraction modalities kit may further comprise at least one sample microslit filter, at least one reference microslit filter, a light source, and a detector. The light source, detector, and signal processing algorithm of the kit comprise elements for trans-illumination of sample and reference microslit filters and for recording, collecting, and comparing their respective optical images and/or diffraction spectra.

In a further example, a kit of the present disclosure may further comprise one or more light source, a detector and an algorithm, for carrying out Raman microscopy and optical microscopy for determining the size and/or composition of components of a sample retained by microslit filters.

The steps of the method described in the various embodiments and examples disclosed herein are sufficient to carry out the methods of the present disclosure. Thus, in an embodiment, a method consists essentially of a combination of the steps of one or more of the methods disclosed herein. In another embodiment, a method consists of such steps.

In the following Statements, various examples of methods, fluidic devices, and kits of the present disclosure are described:

Statement 1. A device (e.g., a fluidic device) comprising: one or more microslit filters, wherein each microslit filter defines a plurality of openings.
Statement 2. A device according to Statement 1, further comprising a first fluidic channel or chamber on a side of the microslit filter and a second fluidic channel or chamber on an opposite side of the microslit filter.
Statement 3. A device according to Statements 1 or 2, where the microslit filter has a thickness from 50 nm to 25 μm.
Statement 4. A device according to any one of the preceding Statements, where the microslit filter has a porosity from 1% to 75%.
Statement 5. A device according to any one of the preceding Statements, where the openings are cubic prisms, rectangular prisms, or trapezoids.
Statement 6. A device according to any one of the preceding Statements, where the openings have a width from 0.5 μm to 15 μm and a length from 5 μm to 100 μm.
Statement 7. A device according to any one of the preceding Statements, where the openings have an aspect ratio from 1:0.33 to 1:200.
Statement 8. A device according to any one of the preceding Statements, where the at least one microslit filter further comprises at least one filtration membrane, one fluidic cavity (e.g., intervening fluidic cavity), and one second filtration membrane.
Statement 9. A device according to any one of the preceding Statements, where the microslit filter further comprises a metal coating.
Statement 10. A device according to any one of the preceding Statements, where the microslit filter is 400 nm thick and has 17% porosity, where the openings are at least 8 μm in width and 50 μm in length, and where the openings have an aspect ratio of at least 1:6.25.
Statement 11. A device according to any one of Statements 1-9, where the microslit filter is 400 nm thick and has 9% porosity, where the openings are 1 μm in width and 50 μm in length, and where the openings have an aspect ratio of 1:50.
Statement 12. A device according to any one of Statements 1-9, where the microslit filter is 400 nm thick and has 9% porosity, where the openings are 0.5 μm in width and 50 μm in length, and where the openings have an aspect ratio of 1:100.
Statement 13. A device according to any one of the preceding Statements, where the microslit filters are functionalized with a non-fouling surface treatment.
Statement 14. A device according to any one of the preceding Statements, where the microslit filters are functionalized to increase interactions between the retained components and the microslit filters.
Statement 15. A device according to any one of the preceding Statements, further comprising a light source and a detector configured to record optical signals of an optical modality.
Statement 16. A device according to any one of the preceding Statements, further comprising a voltmeter, one or more pair of electrodes, and optionally, a function generator, configured to record electrical resistance or impedance of an electronic interrogation.
Statement 17. A device according to any one of the preceding Statements, further comprising one or more pressure sensors configured to record transmembrane pressure of a pressure-dependent modality.
Statement 18. A device according to any one of the preceding Statements, further comprising a flow meter.
Statement 19. A device according to any one of the preceding Statements, further comprising at least one sample microslit filter and at least one reference microslit filter.
Statement 20. A device according to any one of the preceding Statements, further comprising at least one additional microslit filter configured to perform an upstream sample preparation.
Statement 21. A method comprising:
contacting the liquid sample with a microslit filter, such that the components of interest are retained by the microslit filter and undesired components permeate through the microslit filter;
optionally, washing the retained components; and
measuring the quantity of retained components to determine their concentration in the liquid sample.
Statement 22. A method of Statement 21, where measuring the quantity of retained components to determine their concentration in the liquid sample comprises optical imaging, electronic interrogation, optical diffraction, or transmembrane pressure.
Statement 23. A method according to Statements 21 or 22, where the component of interest comprises a biological component.
Statement 24. A method according to any one of Statements 21-23, where the liquid sample is a biofluid sample.
Statement 25. A method according to Statements 21 or 22, where the component of interest comprises a non-biological component.
Statement 26. A method according to any one of Statements 21, 22, 25, or 26, where the liquid sample comprises a food, environmental, or industrial sample.
Statement 27. A method according to any one of Statements 21-26, where the measurement of components is performed as a single instance or as replicate instances.
Statement 28. A method according to any one of Statements 21-27, where the filtering includes one of gravity flow, hydrostatic pressure, pumping, vacuum, centrifugation, gas pressurization, or tangential flow.
Statement 29. A method according to any one of Statements 21-28, where the filtering occurs at a pressure from 10 Pa to 1.0 kPa.
Statement 30. A method according to any one of Statements 21-29, where the method further comprises an upstream sample preparation.
Statement 31. A method according to any one of Statements 21-30, where the method further comprises performing one or more first analytical assay on retained components of interest.
Statement 32. A method according to any one of Statements 21-31, further comprising adding a binding agent to perform a first analytical assay on retained components of interest.
Statement 33. A method according to any one of Statements 21-32, further comprising an optional elution of retained components to perform a second analytical assay.
Statement 34. A method according to any one of Statements 21-33, where the sample comprises a combination of biological and non-biological components.
Statement 35. A method comprising:
contacting the liquid sample with two or more microslit filters;
optionally, washing the retained components; and
measuring the quantity of the two or more populations of retained components to determine their concentration in the liquid sample.
Statement 36. A method according to Statement 35, where measuring the quantity of retained components to determine their concentration in the liquid sample comprises optical imaging, electronic interrogation, or transmembrane pressure.
Statement 37. A method according to Statements 35 or 36, where the component of interest comprises a biological component, a non-biological component, or combinations thereof.
Statement 38. A method according to any one of Statements 35-37, where the liquid sample is a biofluid sample, a food sample, an environmental sample, an industrial sample, or combinations thereof.
Statement 39. A method according to any one of Statements 35-38, where contact with the two or more microslit filters further comprises fluidic contact of the liquid sample with two or more first filtration membranes, two or more fluidic cavities, and two or more second filtration membranes.
Statement 40. A method according to any one of Statements 35-39, where the measurement of the two or more populations of components is performed as a single instance or as replicate instances for each component population.
Statement 41. A method according to any one of Statements 35-40, where the filtering includes one of gravity flow, hydrostatic pressure, pumping, vacuum, centrifugation, gas pressurization, or tangential flow.
Statement 42. A method according to any one of Statements 35-41, where the filtering occurs at a pressure from 10 Pa to 1.0 kPa.
Statement 43. A method according to any one of Statements 35-42, where the method further comprises an upstream sample preparation.
Statement 44. A method according to any one of Statements 35-43, where the liquid sample is whole blood and the measured components comprise leukocytes, erythrocytes, and platelets, and a complete blood cell count is performed.
Statement 45. A method according to any one of Statements 35-44, where the method further comprises performing one or more first analytical assay on retained components.
Statement 46. A method according to any one of Statements 35-45, further comprising adding a binding agent to perform a first analytical assay on retained components.
Statement 47. A method according to any one of Statements 35-46, where the liquid sample is whole blood and the measured components comprise leukocytes, erythrocytes, and platelets, and the method further comprises using binding agents and first analytical assays to perform a complete blood cell count with differentiation.
Statement 48. A method according to any one of Statements 21-34 or 35-45, where the method further comprises performing a microplastic particle test on a liquid sample.
Statement 49. A method according to any one of Statements 21-34 or 35-45, where the method further comprises performing a biological component production test.
Statement 50. A method according to any one of Statements 35-48, further comprising an optional elution of retained components to perform a second analytical assay.
Statement 51. A kit comprising one or more device of the present disclosure (e.g., one or more device of any one of claims 1-20) and/or one or more reagents (e.g., one or more reagents of the present disclosure) for carrying out a method of the present disclosure (e.g., a method of any one of claims 21-50).
Statement 52. A kit according to Statement 51, where the kit further comprises instructions for use of the one or more device (e.g., one or more device according to any one of Statements 1-20) and/or one or more reagents (e.g., one or more reagents of the present disclosure).
Statement 53. A kit according to Statements 51 or 52, where the kit further comprises instructions for carrying out the methods of the present disclosure (e.g., a method according to any one of Statements 21-50).
Statement 54. A kit according to any one of Statements 51-53, where the one or more reagents are selected from binding agents, buffers, solutions, and/or reagents for enzymes and the like, model components in solution (of known concentration), and combinations thereof.
Statement 55. A kit according to any one of Statements 51-54, where the binding agents are selected from affinity moieties, detection agents, and combinations thereof.
Statement 56. A kit according to any one of Statements 51-55, where the kit further comprises a light source and/or a detector.
Statement 57. A kit according to any one of Statements 51-56, where the kit further comprises a flow meter.
Statement 58. A kit according to any one of Statements 51-57, where the kit further comprises a voltmeter, one or more pair of electrodes, and optionally, a function generator.
Statement 59. A kit according to any one of Statements 51-58, where the kit further comprises one or more pressure sensors.
Statement 60. A kit according to any one of Statements 51-59, where the kit further comprises a signal processing algorithm for optical, electronic, and/or pressure signals.
Statement 61. A kit according to any one of Statements 51-60, where the kit further comprises one or more algorithms for calculating concentration of components from a liquid sample.
Statement 62. A kit according to any one of Statements 51-61, where the kit further comprises one or more algorithms for registering retained components on microslit filters and determine the size and/or composition of such components.

In the following Statements, various examples of methods, fluidic devices, and kits of the present disclosure are described:

Statement 1A. A method comprising: contacting a liquid sample comprising a plurality of components with a microslit filter, such that one or more components of interest are retained by the microslit filter and undesired components permeate through the microslit filter; optionally, washing the retained components of interest; and measuring the quantity of retained components of interest to determine the concentration of the retained components of interest in the liquid sample.
Statement 2A. A method according to Statement 1A, where measuring the quantity of retained components of interest comprises optical imaging, electronic interrogation, optical diffraction, or transmembrane pressure.
Statement 3A. A method according to Statements 1A or 2A, where the one or more components of interest comprises a biological component.
Statement 4A. A method according to any one of Statements 1A-3A, where the liquid sample is a biofluid sample.
Statement 5A. A method according to any one of Statements 1A-4A, where the one or more components of interest comprises a non-biological component.
Statement 6A. A method according to any one of Statements 1A-5A, where the liquid sample comprises a food, environmental, or industrial sample.
Statement 7A. A method according to any one of Statements 1A-6A, where the measuring the quantity of retained components of interest is performed as a single instance or as replicate instances.
Statement 8A. A method according to any one of Statements 1A-7A, where the contacting a liquid sample with a microslit filter, such that one or more components of interest are retained by the microslit filter and undesired components permeate through the microslit filter includes gravity flow, hydrostatic pressure, pumping, vacuum, centrifugation, gas pressurization, or tangential flow.
Statement 9A. A method according to any one of Statements 1A-8A, where the contacting a liquid sample with a microslit filter, such that one or more components of interest are retained by the microslit filter and undesired components permeate through the microslit filter occurs at a pressure from 10 Pa to 1.0 kPa.
Statement 10A. A method according to any one of Statements 1A-9A, further comprising preparing one or more upstream sample.
Statement 11A. A method according to any one of Statements 1A-10A, further comprising performing one or more first analytical assay on the one or more retained components of interest.
Statement 12A. A method according to any one of Statements 1A-11A, further comprising adding a binding agent and performing a first analytical assay on the one or more retained components of interest.
Statement 13A. A method according to any one of Statements 1A-12A, further comprising eluting the one or more retained components of interest and performing a second analytical assay.
Statement 14A. A method according to any one of Statements 1A-13A, where the sample comprises a combination of biological and non-biological components.
Statement 15A. A method according to any one of Statements 1A-14A, where the method further comprises performing a microplastic particle test on a liquid sample.
Statement 16A. A method according to any one of Statements 1A-15A, wherein the method further comprises performing a biological component production test.
Statement 17A. A method comprising: contacting a liquid sample comprising a plurality of components with two or more microslit filters, such that two or more populations of components of interest are retained by the two or more microslit filters and undesired components permeate through the two or more microslit filters; optionally, washing the retained components; and measuring the quantity of the two or more populations of retained components to determine the concentration of the two or more populations of retained components in the liquid sample.
Statement 18A. A method according to Statement 17A, where measuring the quantity of the two or more populations of retained components to determine the concentration of the two or more populations of retained components in the liquid sample comprises optical imaging, electronic interrogation, or transmembrane pressure.
Statement 19A. A method according to Statements 17A or 18A, where the components of interest comprise a biological component, a non-biological component, or combinations thereof.
Statement 20A. A method according to any one of Statements 17A-19A, where the liquid sample is a biofluid sample, a food sample, an environmental sample, an industrial sample, or combinations thereof.
Statement 21A. A method according to any one of Statements 17A-20A, where contacting a liquid sample with two or more microslit filters comprises fluidic contact of the liquid sample with two or more first filtration membranes, two or more fluidic cavities, and two or more second filtration membranes.
Statement 22A. A method according to any one of Statements 17A-21A, where measuring the quantity of the two or more populations of retained components is performed as a single instance or as replicate instances for each component population.
Statement 23A. A method according to any one of Statements 17A-22A, where contacting a liquid sample comprising a plurality of components with two or more microslit filters, such that two or more populations of components of interest are retained by the two or more microslit filters and undesired components permeate through the two or more microslit filters includes gravity flow, hydrostatic pressure, pumping, vacuum, centrifugation, gas pressurization, or tangential flow.
Statement 24A. A method according to any one of Statements 17A-23A, where contacting a liquid sample comprising a plurality of components with two or more microslit filters, such that two or more populations of components of interest are retained by the two or more microslit filters and undesired components permeate through the two or more microslit filters occurs at a pressure from 10 Pa to 1.0 kPa.
Statement 25A. A method according to any one of Statements 17A-24A, further comprising preparing one or more upstream sample.
Statement 26A. A method according to any one of Statements 17A-26A, where the liquid sample is whole blood and the plurality of components comprise leukocytes, erythrocytes, and platelets.
Statement 27A. A method according to Statement 26A, further comprising performing a complete blood cell count.
Statement 28A. A method according to any one of Statements 17A-27A, further comprising performing one or more first analytical assay on the two or more populations of retained components of interest.
Statement 29A. A method according to any one of Statements 17A-28A, further comprising adding a binding agent and performing a first analytical assay on the two or more populations of retained components of interest.
Statement 30A. A method according to any one of Statements 17A-29A, where the liquid sample is whole blood and the measured components comprise leukocytes, erythrocytes, and platelets, and the method further comprises using binding agents and performing first analytical assays, such that a complete blood cell count with differentiation results.
Statement 31A. A method according to any one of Statements 17A-30A, further comprising performing a microplastic particle test on the liquid sample.
Statement 32A. A method according to any one of Statements 17A-31A, further comprising performing a biological component production test.
Statement 33A. A method according to any one of Statements 17A-32A, further comprising eluting the two or more populations of retained components to perform a second analytical assay.
Statement 34A. A device comprising one or more microslit filters, wherein each microslit filter comprises a plurality of openings.
Statement 35A. A device according to Statement 34A, further comprising a first fluidic channel or chamber on a side of the microslit filter and a second fluidic channel or chamber on an opposite side of the microslit filter.

Statmeent 36A. A device according to Statements 34A or 35A, wherein the microslit filter has a thickness from 50 nm to 25 μm.

Statement 37A. A device according to any one of Statements 34A-36A, wherein the microslit filter has a porosity from 1% to 75%.
Statement 38A. A device according to any one of Statements 34A-37A, where the openings are cubic prisms, rectangular prisms, or trapezoids.
Statement 39A. A device according to any one of Statements 34A-38A, where the openings have a width from 0.5 μm to 15 μm and a length from 5 μm to 100 μm.
Statement 40A. A device according to any one of Statements 34A-39A, where the openings have an aspect ratio from 1:0.33 to 1:200.
Statement 41A. A device according to any one of Statements 34A-40A, where the at least one microslit filter further comprises at least one filtration membrane, one fluidic cavity, and one second filtration membrane, wherein the one fluidic cavity is an intervening fluidic cavity.
Statement 42A. A device according to any one of Statements 34A-41A, where the microslit filter further comprises a metal coating.
Statement 43A. A device according to any one of Statements 34A-42A, where the microslit filter is 400 nm thick and has 17% porosity, where the openings are at least 8 μm in width and 50 μm in length and have an aspect ratio of at least 1:6.25.
Statement 44A. A device according to any one of Statements 34A-43A, where the microslit filter is 400 nm thick and has 9% porosity, where the openings are 1 μm in width and 50 μm in length and have an aspect ratio of 1:50.
Statement 45A. A device according to any one of Statements 34A-44A, where the microslit filter is 400 nm thick and has 9% porosity, where the openings are 0.5 μm in width and 50 μm in length and have an aspect ratio of 1:100.
Statement 46A. A device according to any one of Statements 34A-45A, further comprising one or more non-fouling surfaces.
Statement 47A. A device according to any one of Statements 34A-46A, where the microslit filter is further configured to increase interactions between one or more retained components and the microslit filters.
Statement 48A. A device according to any one of Statements 34A-47A, further comprising a light source and a detector configured to record optical signals of an optical modality.
Statement 49A. A device according to Statement 48A, where the optical modality is an intensity of a diffraction pattern.
Statement 50A. A device according to Statement 48A, where the light source and the detector are configured to record optical signals of an optical modality by performing Fourier-transformed infrared spectroscopy or Raman spectroscopy.
Statement 51A. A device according to any one of Statements 34A-50A, further comprising an electrical interrogator configured to record an electrical property.
Statement 52A. A device according to Statement 51A, where the electrical interrogator comprises a voltmeter, or one or more pairs of electrodes.
Statement 53A. A device according to Statements 51A or 52A, where the electrical interrogator comprises a function generator.
Statement 54A. A device according to any one of Statements 51A-53A, where the electrical property is conductivity, resistance, impedance, current, or voltage.
Statement 55A. A device according to any one of Statements 34A-54A, further comprising one or more pressure sensors configured to record trans-membrane pressure of a pressure-dependent modality.
Statement 56A. A device according to Statement 55A, where the pressure-dependent modality is a trans-membrane pressure drop.
Statement 57A. A device according to any one of Statements 34A-56A, further comprising a flow meter.
Statement 58A. A device according to any one of Statements 34A-57A, further comprising at least one sample microslit filter and at least one reference microslit filter.
Statement 59A. A device according to any one of Statements 34A-58A, further comprising at least one additional microslit filter configured to perform an upstream sample preparation.
Statement 60A. A device according to any one of Statements 34A-59A, further comprising a fouling sensor configured to detect a level of fouling on at least one portion of the microslit filter.
Statement 61A. A device according to any one of Statements 34A-60A, further comprising an optical imaging device configured to record an optical imaging modality.
Statement 62A. A device according to Statement 61, where the optical imaging modality is surface plasmon resonance, plasmon-enhanced fluorescence, surface, enhanced fluorescence, or surface-enhanced Raman spectroscopy.
Statement 63A. A device according to any one of Statements 34A-62A, where the device is a fluidic device.
Statement 64A. A kit comprising one or more device according to any one of Statements 34A-63A and/or one or more reagents of the present disclosure.
Statement 65A. A kit according to Statement 64A, where the kit further comprises instructions for use of the one or more device and/or one or more reagents.
Statement 66A. A kit according to Statements 64A or 65A, where the kit further comprises instructions for carrying out the methods of the present disclosure (e.g., a method according to any one of Statements 1A-16A).
Statement 67A. A kit according to Statements 64A or 65A, where the kit further comprises instructions for carrying out the methods of the present disclosure (e.g., a method according to any one of Statements 17A-33A).
Statement 68A. A kit according to any one of Statements 64A-67A, where the one or more reagents are selected from binding agents, buffers, solutions, and/or reagents for enzymes and the like, model components in solution (of known concentration), and combinations thereof.
Statement 69A. A kit according to any one of Statements 64A-68A, where the binding agents are selected from affinity moieties, detection agents, and combinations thereof.
Statement 70A. A kit according to any one of Statements 64A-69A, where the kit further comprises a light source and/or a detector.
Statement 71A. A kit according to any one of Statements 64A-70A, where the kit further comprises a flow meter.
Statement 72A. A kit according to any one of Statements 64A-71A, where the kit further comprises a voltmeter, one or more pair of electrodes, and optionally, a function generator.
Statement 73A. A kit according to any one of Statements 64A-72A, further comprising one or more pressure sensors.
Statement 74A. A kit according to any one of Statements 64A-73A, further comprising a signal processing algorithm for optical, electronic, and/or pressure signals.
Statement 75A. A kit according to any one of Statements 64A-74A, further comprising one or more algorithms for calculating concentration of components from a liquid sample.
Statement 76A. A kit according to any one of Statements 64A-75A, further comprising one or more algorithms for registering retained components on microslit filters and determine the size and/or composition of such components.

The following examples are presented to illustrate the present disclosure. They are not intended to be limiting in any matter.

### Example 1

This example provides a description of examples of devices and methods of the present disclosure, as well as an exemplary method and use of the present disclosure for retaining and measuring a biological component from a biological liquid sample to perform a blood cell count.

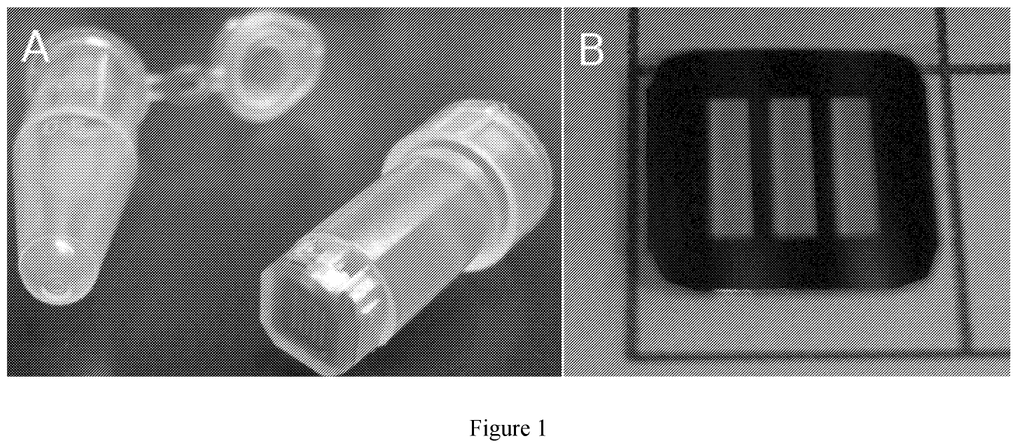

FIG. 1A shows a representative fluidic device incorporating a microslit filter, wherein the microslit filter is integrated into a centrifuge tube insert fluidic device for dead-end (normal) flow filtration purposes. FIG. 1B shows a representative microslit filter comprising 400 nm thick silicon nitride membranes, with three 0.7×3 mm suspended membranes, disposed on a silicon substrate of 5.4×5.4 mm and 0.3 mm thickness. The three 0.7×3 mm silicon nitride membranes further comprise a plurality of 8×50 μm openings patterned and etched through the 400 nm thick silicon nitride membranes. Conventional photolithography, reactive ion etching, and wet chemistry through-wafer etching were used to fabricate such microslit filters.

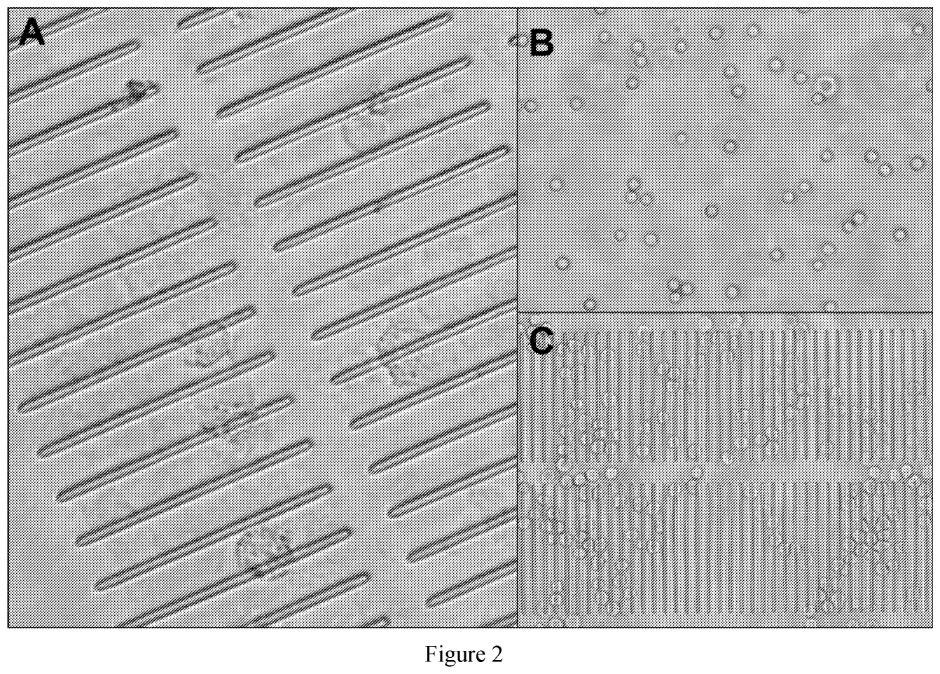

FIG. 2A shows a similar microslit filter as shown in FIG. 1B, after contact with a 0.1 mL solution of a biological liquid sample, comprising whole blood diluted to 0.1% (v/v) in phosphate-buffered saline (PBS) formulated at 270 mOsm, pH 7.4. Such a dilution represents one of the optional pre-treatment steps of the present disclosure. The biological components (e.g., cells) retained by this microslit filter are consistent with white blood cells (e.g., leukocytes). The fluidic device used for this filtration comprised a microslit filter similar to that shown in FIG. 1A in all respects except that the microslit filter of FIG. 2A comprised membranes with 1×50 μm openings. FIG. 2B shows a second filter, comprising nanoporous silicon nitride (100 nm thickness, 60 nm average diameter pores, 20% porosity), after contact with a 0.1 mL volume of the flow-through from the filtration performed using the 1×50 μm microslit filter of FIG. 2A. The cells retained on the nanoporous filter from the flow-through are consistent with red blood cells (e.g., erythrocytes). FIG. 2C shows a third filter after contact with a 0.1 mL volume of the flow-through from the filtration performed using the 1×50 μm microslit filter of FIG. 2A. The cells retained on this third filter are consistent with red blood cells (e.g., erythrocytes). The fluidic device used for this filtration comprised a microslit filter similar to that shown in FIG. 1A in all respects except that the microslit filter of FIG. 2C comprised membranes with 0.5×50 μm openings. All filtrations shown in FIGS. 2A-C were done by centrifugation at 600×G for 10 minutes, then the microslit filters were extracted from fluidic devices (e.g., for an ex situ measurement), and imaged on a confocal light microscope at 40× magnification.

### Example 2

This example provides a description of the optical diffraction modality of the present disclosure for a model non-biological component retained and measured from a liquid sample.

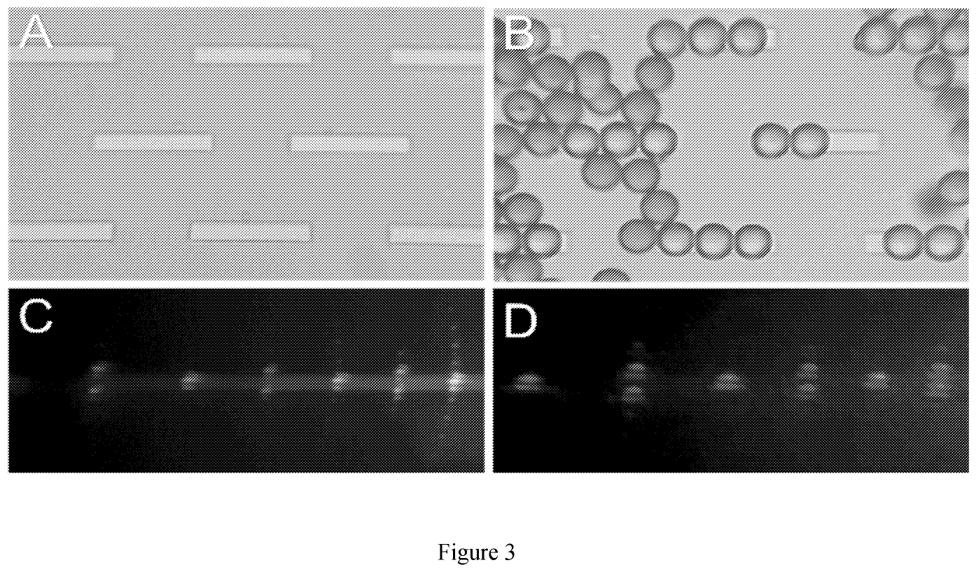

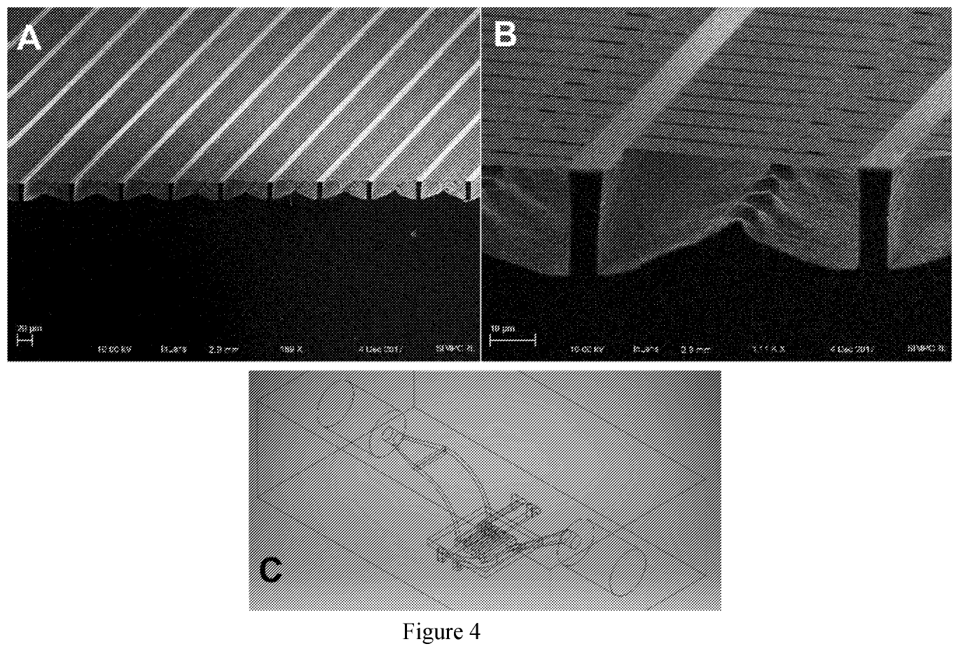

FIG. 3A shows the native surface of the microslit filter of FIG. 1B via light microscopy). FIG. 3B demonstrates an identical filter retaining 16 μm polystyrene beads. FIGS. 3C, and 3D demonstrate the corresponding diffraction patterns of the two former microslit filters, where FIG. 3C is the reference image for the diffraction pattern shift observed by microslit occlusion in FIG. 3D.

### Example 3

This example provides a description of the microslit filter-fluidic cavity monolithic structure of the present disclosure.

FIG. 4A shows an electron microscopy image of an intervening fluidic cavity fabricated by transmembrane etching through microslit openings. Fluidic connection to this intervening fluidic cavity is through the microslit openings, within the resultant filtration membranes. FIG. 4B shows one potential scheme for fluidically connecting multiple filtration membranes and intervening fluidic cavities.

FIG. 4C shows a fluidic rendering of a device for fluidically addressing the membrane system from FIGS. 4A and 4B. The plurality of fluidic cavities is interconnected in series by a gasket interface.

### Example 4

This example provides a description of a tangential flow fluidic device incorporating microslit filters.

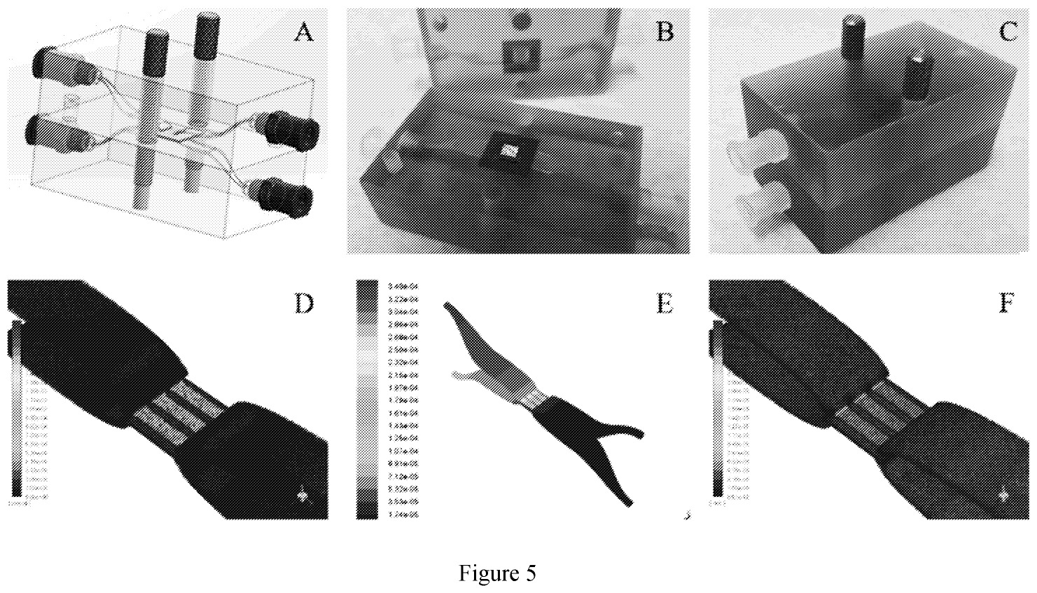

FIG. 5 shows a panel of images describing the design and modeling of an exemplary tangential flow fluidic device for incorporating microslit filters (FIG. 5A). A 3D-printed prototype with polycarbonate body and elastomer gasket (dark center region) is shown after partially assembly of the device with a microslit filter installed in the centering gasket (FIG. 5B), and after complete assembly (FIG. 5C). Using ANSYS software, surface velocity (FIG. 5D), system pressure (FIG. 5E), and shear stress (FIG. 5F), were modeled to verify suitability for utilizing whole blood in the device.

This figure demonstrates the critical importance of membrane thickness on operating pressurization. As this simulation shows, the transmembrane pressure differential (TMP) increases proportionally to the membrane thickness. In this simulation, the TMP of a representative 50 nm thick membrane is very low (˜12 Pa). As the membrane thickness increases to 5 μm and then 50 μm, there is a significant increase in TMP from ˜20 Pa to ˜65 Pa, respectively. Thus, low membrane thickness (e.g., ≤1 μm thick) enables far lower operating pressures relative to thicker membranes (e.g., ≥5 μm thick).

### Example 5

This example provides a description of the pressurization within an exemplary tangential flow fluidic device incorporating microslit filters.

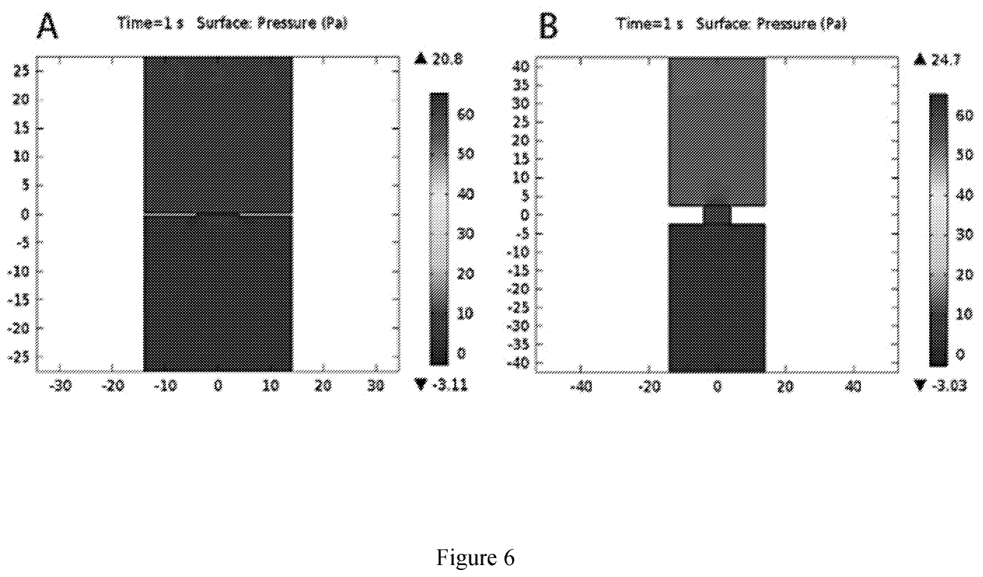

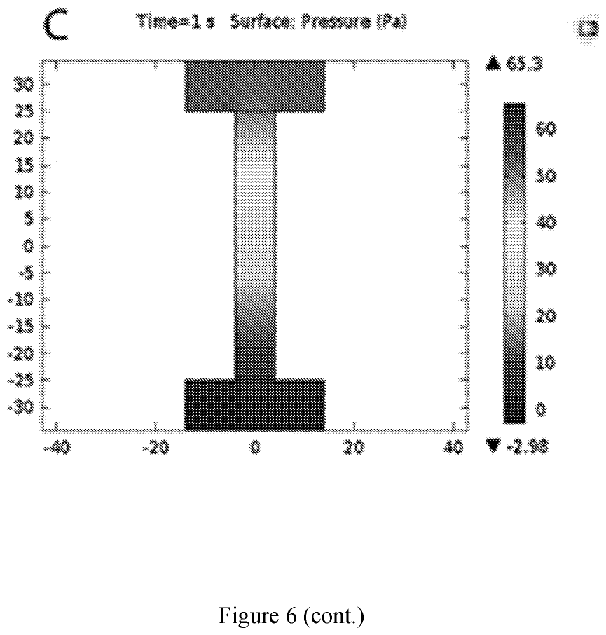

FIG. 6A-C demonstrate the results of a pressure drop analysis which evaluated microslit height (e.g., thickness of a filtration membrane) for a suspension of cells to minimize sheer stress and subsequent cell damage. FIG. 6A demonstrates a negligible 20.8 Pa pressure drop for the exemplary 400 nm thick microslit filters described in the present disclosure. Increasing membrane thickness to 5 and 50 μm respectively (FIGS. 6B and 6C) yields a concomitant decrease in pressure through the membrane of 25 Pa and 65 Pa respectively.

### Example 6

This example provides a description of a microplastic water test by one exemplary method of the present disclosure.

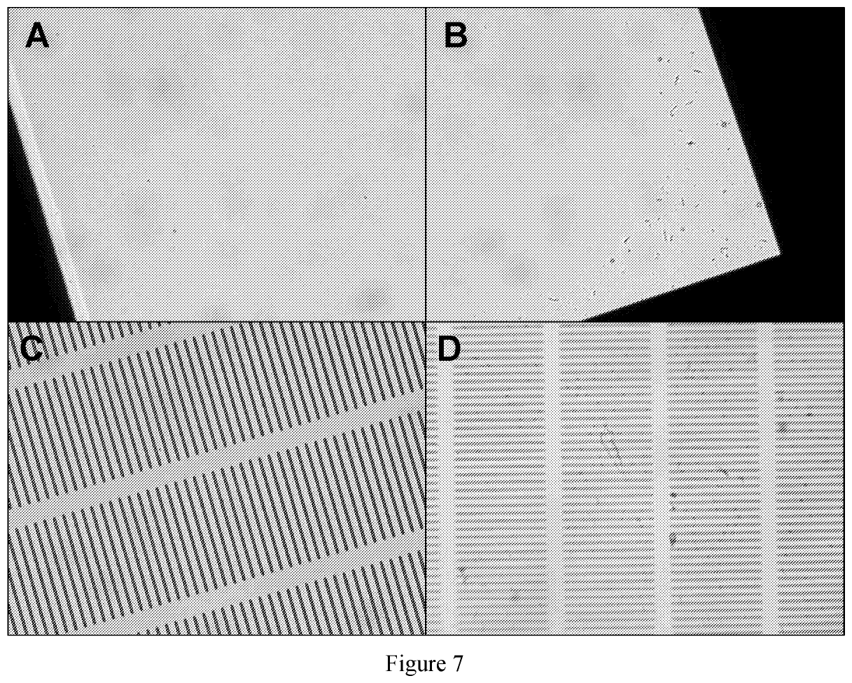

FIG. 7 shows the resulting phase contrast microscopy images taken at 40× magnification of the resultant membrane filters after filtering two different water samples. The fluidic devices where similar to those shown and used in Examples 1-2. In one case, centrifuge spin columns incorporating nanoporous silicon nitride (NPN) filters with 50 nm average pore diameter were used to retain particles from either (A) MilliQ water (18.3 mOhm resistivity, 0.2 μm filtered), or (B) Tap water with no pretreatment. The water samples were centrifuged at 1,000×G for 10 minutes and 2 mL water samples were passed through each fluidic device. Microslit filter-incorporating fluidic devices were used to retain particles from either (C) MilliQ water (18.3 mOhm resistivity, 0.2 μm filtered), or (D) Tap water with no pretreatment. The water samples were centrifuged at 1,000×G for 10 minute and 2 mL water samples were passed through each fluidic device. After centrifugation, the membranes from all devices were extracted (e.g., for an ex situ measurement) and imaged via phase contrast light microscopy (all images 40×). Retained particles are consistent with both inorganic and organic composition, based on observed morphology.

### Example 7

This example provides a description of a method and use of the present disclosure for optional elution following selective retention of a model non-biological component from a liquid sample.

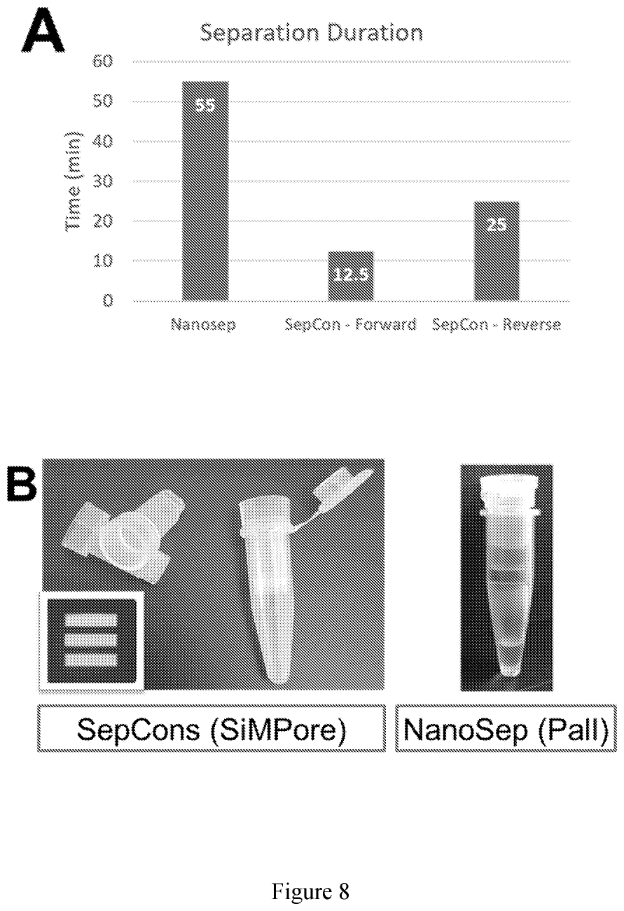

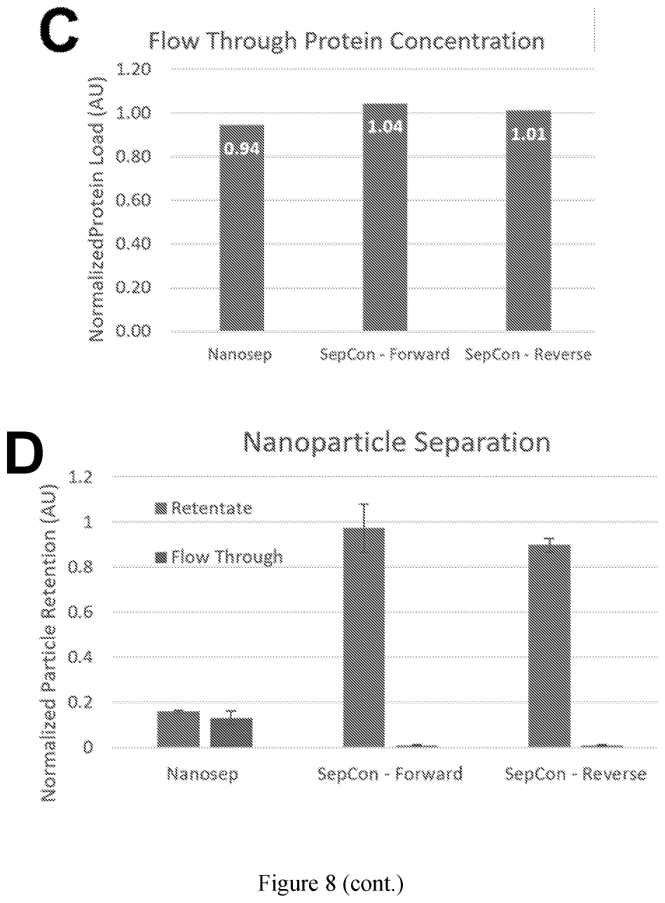

FIG. 8 shows the results from the selective retention by a microslit filter of a polymeric particle. A suspension of fluorescent red polystyrene particles (2.0 μm average diameter) was prepared at 1×10\hat{ }6 particles/mL in PBS with 0.5 mg/mL bovine serum albumin (BSA). 400 μL of this solution was centrifuged through various fluidic devices until the volume was passed and the time to complete volume passage was recorded. Fluidic devices with 1×50 slit openings were used (FIG. 8B; labeled as “SepCon”), wherein one set of fluidic devices operated in the “Forward” mode similar to this shown in FIGS. 1-2, while a second set of fluidic devices operated in a “Reverse” mode, wherein the flow moves in an opposite flow pattern to that in the Forward mode. The same particle solution was also centrifuged through a commercial polymeric filter spin column (Pall NanoSep®, 300 kDa cut-off Omega membranes). FIG. 8A demonstrates the higher permeability of microslit filters versus polymeric filters, as evidenced by the significantly reduced filtration time for the microslit filter-containing spin column to pass the entire 400 μL volume (12.5-25 minutes versus 50 minutes). FIG. 8C demonstrates essentially complete recovery of the BSA in the filtrate produced by the microslit filter-containing spin columns in either Forward or Reverse modes, whereas 6% protein loss was observed to the polymeric filter. FIG. 8D shows both higher particle retention and particle recovery (using an optional elution method of the present disclosure) for microslit filter-containing spin columns relative to the commercial polymeric filter spin column.

### Example 8

This example provides a characterization of the filtration properties using model non-biological components for a representative nanoporous filter.

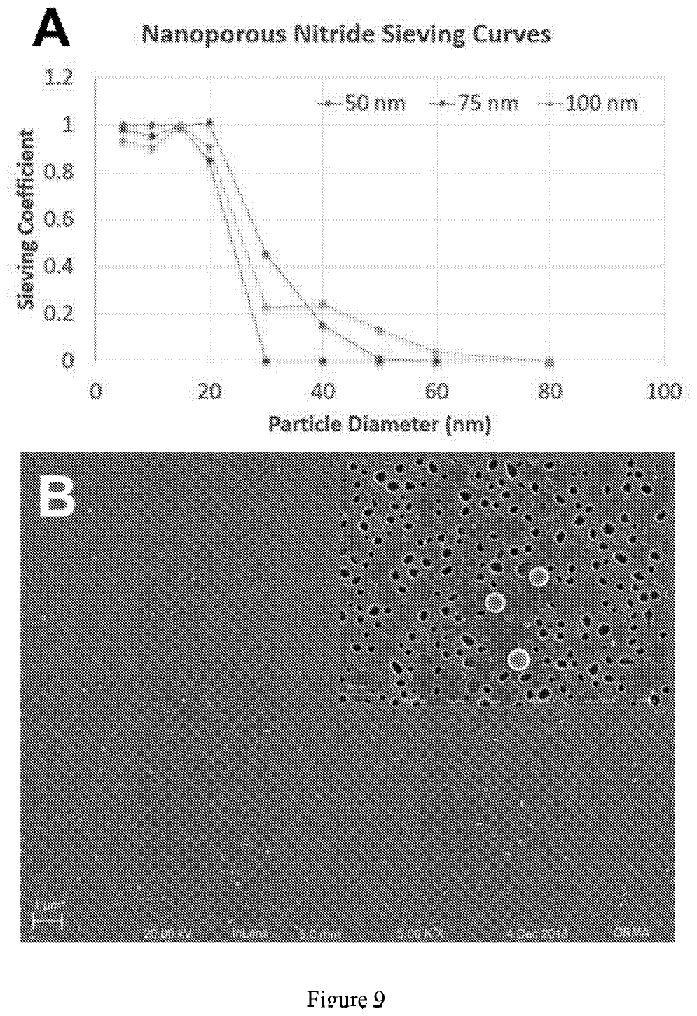

FIG. 9A shows particle retention and permeation results for three different nanoporous filters (50 nm, 75 nm, and 100 nm thickness, each with its own corresponding average pore diameter (˜23 nm, ˜33 nm, and ˜45 nm, respectively). For each data point in FIG. 9A, a distinct population of gold nanoparticles (with distinct average diameter) were dissolved in water and then passed through each nanoporous filter via centrifugation. The amount of gold particles retained and permeated was measured and results are plotted as percent transmission (e.g., sieving coefficient). FIG. 9B shows a representative 100 nm-thick nanoporous filter retaining 100 nm-average diameter polystyrene particles that was imaged by scanning electron microscopy following the filtration. The nanoporous filter was extracted from the spin column used for this filtration, thus this example demonstrates an ex situ analytical assay of the present disclosure.

### Example 9

This example provides a characterization of the filtration properties using non-biological components for two representative microslit filters.

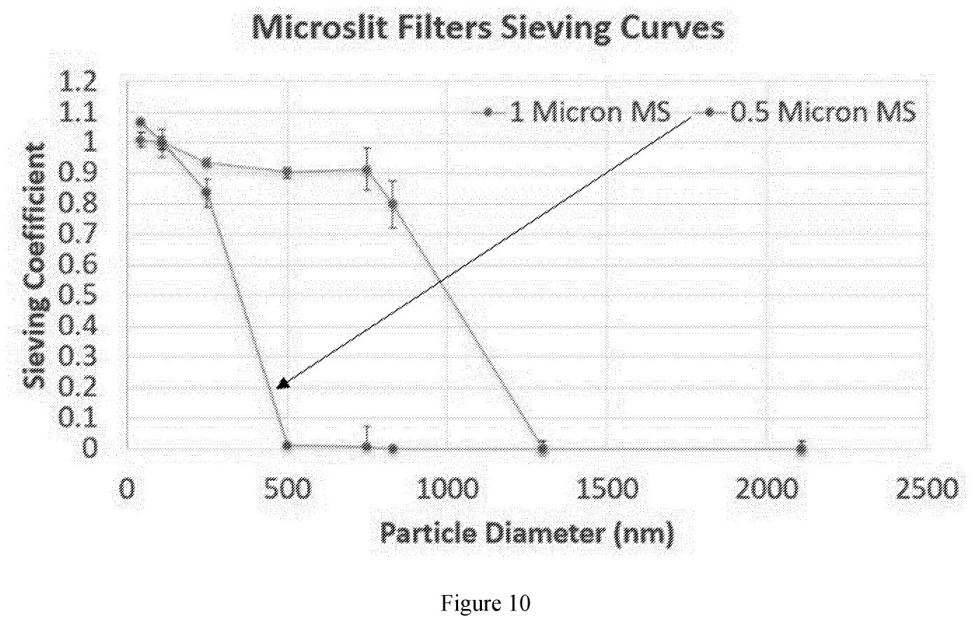

FIG. 10 shows particle retention and permeation results for two different microslit filters with 0.5×50 μm and 1×50 μm slit openings (both with 400 nm thick silicon nitride membranes). For each data point shown in FIG. 10, a distinct population of fluorescent polystyrene particles (with distinct average diameter) were dissolved in PBS to 1×10\hat{ }6 particles/mL and then passed through each microslit filter via centrifugation. The amount of polystyrene particles retained and permeated was measured and results are plotted as percent transmission (e.g., sieving coefficient).

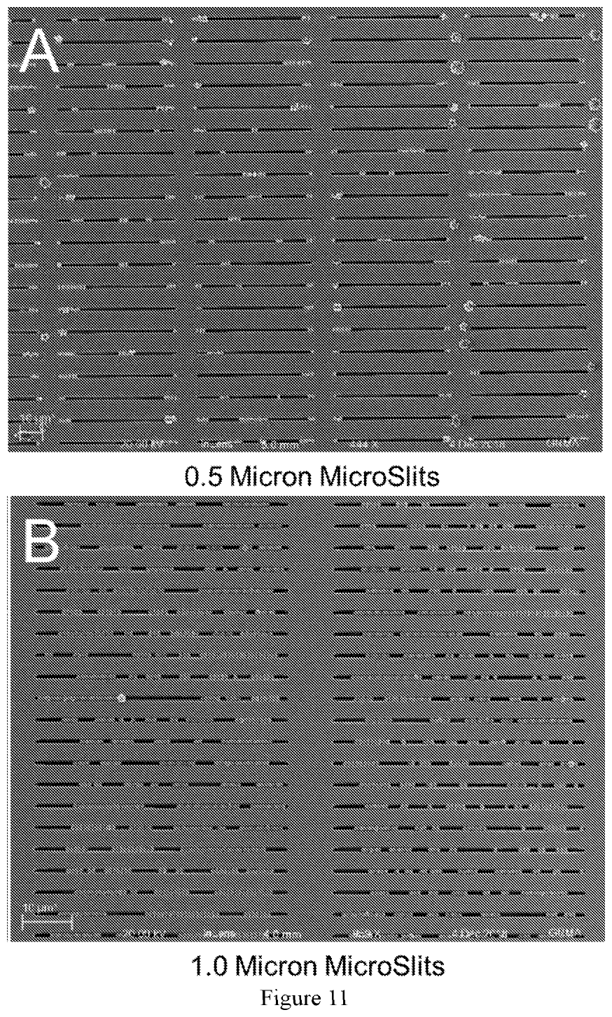

Representative 0.5×50 μm slit opening microslit filters (FIG. 11A) and 1×50 μm slit opening Microslit filters (FIG. 11B) are shown, as imaged by scanning electron microscopy following filtration of particles and extraction of the microslit filters from fluidic devices. Thus, this example represents an ex situ measurement. Solutions of either 850 nm or 1.25 μm diameter polystyrene particles dissolved in PBS were passed via centrifugation through either 0.5×50 μm or 1×50 μm slit opening microslit filters, respectively, prior to extraction and imaging.

### Example 10

This example provides a description of an exemplary use of the transmembrane pressure-dependent method of the present disclosure using model non-biological components and a representative microslit filter.

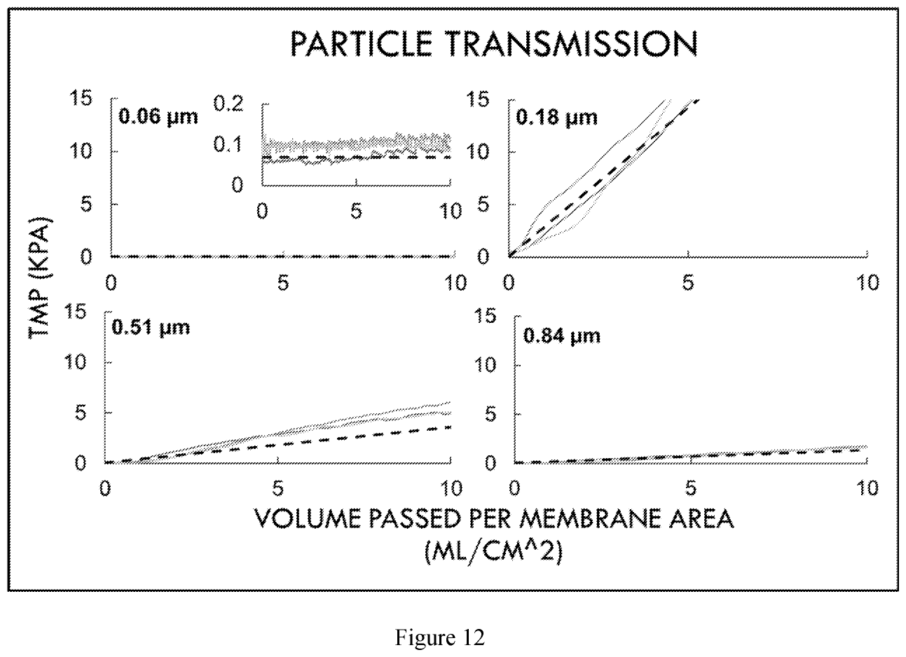

FIG. 12 shows particle retention and permeation results for a microslit filter with 0.2×10 μm slit openings (200 nm thick silicon nitride membranes). For each set of data shown in FIG. 12, a distinct population of fluorescent polystyrene particles (with distinct average diameter) were used. Particles were passed via normal flow filtration. The number of polystyrene particles retained and permeated was measured and results are plotted as percent transmission (e.g., sieving coefficient). Particles were diluted in 0.1 M carbonate buffer (pH 9.4) with 0.01% v/v Tween-20. The buffer was prepared with ultra-pure water and pre-filtered through a 0.2 μm surfactant-free cellulose acetate syringe filter. All particles were diluted to a final concentration of 0.003% w/v and sonicated for 15 minutes in an ultrasonic bath immediately before use.

The particle filtration experiments were performed in a normal flow, constant flux configuration where TMP was monitored relative to atmospheric reference for all samples passed. The microslit filters were incorporated into spin columns (per FIGS. 1-2), which were connected to a PEEK adapter for flow and pressure recording. A USB output pressure transducer (Omega PX409) monitored changes in transmembrane pressure in real-time. Flow was supplied by a syringe pump (Harvard Apparatus PhD 2000); for all experiments a flux of 0.8 mL cm−2 min−1 was used. The transmembrane pressure rise was most pronounced as the particle size approached the narrow width of the microslit filters' openings.

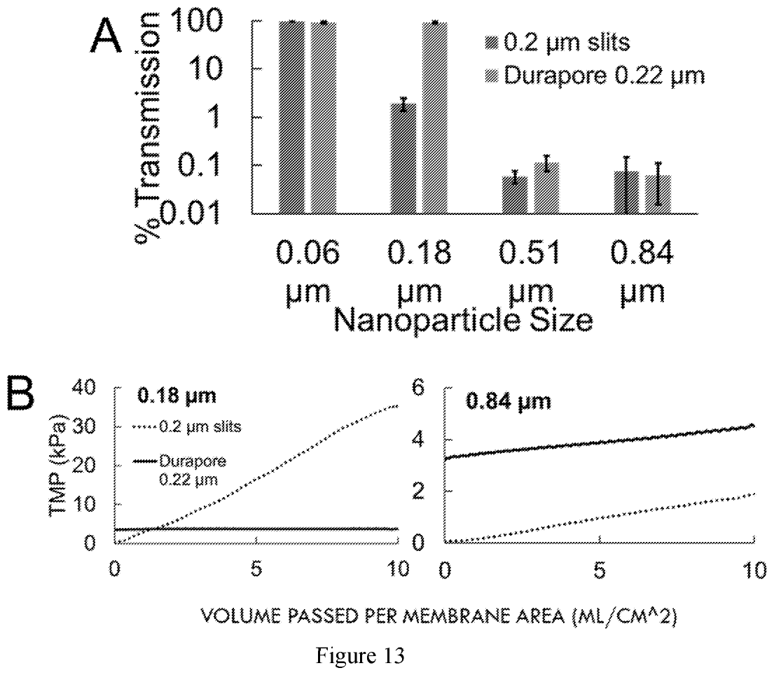

FIG. 13 shows (A) transmission of various nanoparticle sizes through both the 0.2 μm slit membrane and a Durapore 0.22 μm PVDF membrane as a comparison. (B) Difference in fouling between the 0.2 μm slit membrane and the Durapore membrane. Lines shown are the average of 3 filtration runs.

### Example 11

This example provides a description of an ex situ analytical assay of the present disclosure, with optical microscopy used to register components and infrared spectroscopy used to identify the composition of two types of model non-biological components (e.g., polymethylmethacrylate and polyethylene particles retained by microslit filters).

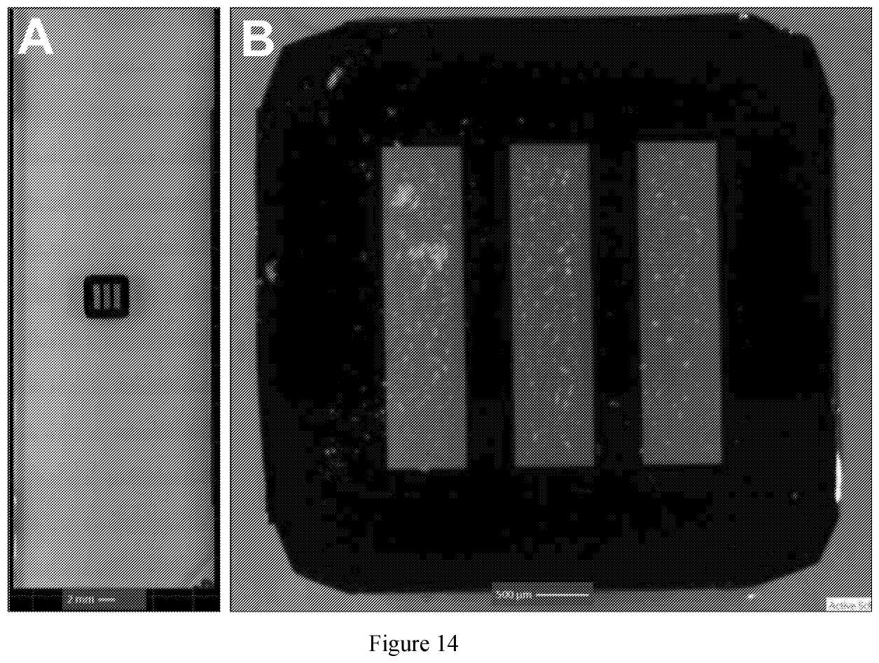

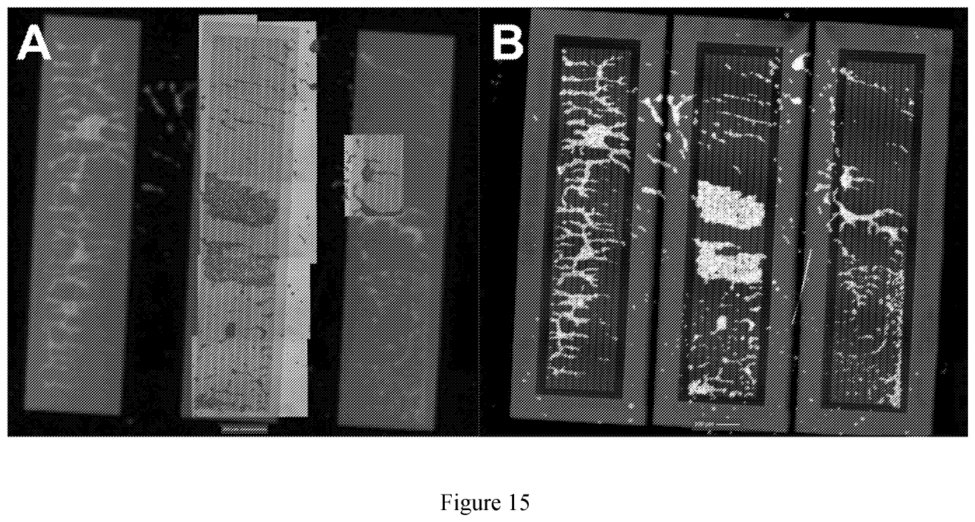

FIG. 14 shows a 0.5×50 μm slit opening microslit filter used to filter a solution of polymeric particles that ranged in size from 1.0 to 15 μm diameter and comprising three distinct compositions (polystyrene, polyethylene, and polymethylmethacrylate). The particles were dissolved to a concentration of 1.0 μg/mL in PBS pH 7.4. Centrifugation was used (600 XG, 20 minutes) to filter 500 μL solution samples. An optional wash step was performed by passing 500 μL of MillliQ water through the spin columns (centrifuged as before). Each microslit filter was then extracted from the spin columns and potted onto MirrIR slides (aluminum coated glass slides; a.k.a. “Kelvey slides”) using Norland 68, optical-grade, UV-cured adhesive. This potting process provided an infrared (IR) reflective background for subsequent analyses.

FIG. 14A shows images of the microslit filter affixed to the MirrIR slide after Norland 68 bonding. FIG. 14B shows a phase contrast light microscopy image of the Microslit filter in 14A at higher magnification, enabling resolution of the polymethylmethacrylate (PMMA) particles retained on the microslit filter.

FIG. 15A shows a phase contrast light microscopy image of a microslit filter with retained polyethylene (PE) particles. FIG. 15B shows the same region of interest as in FIG. 15A, with equal magnification, using an Agilent 8700 LDIR at 1790 cm−1; 8 min, 1 μm pixel size. These data demonstrate a strong IR signature of the microplastic particles retained on the membrane surface relative to standard light microscopy.

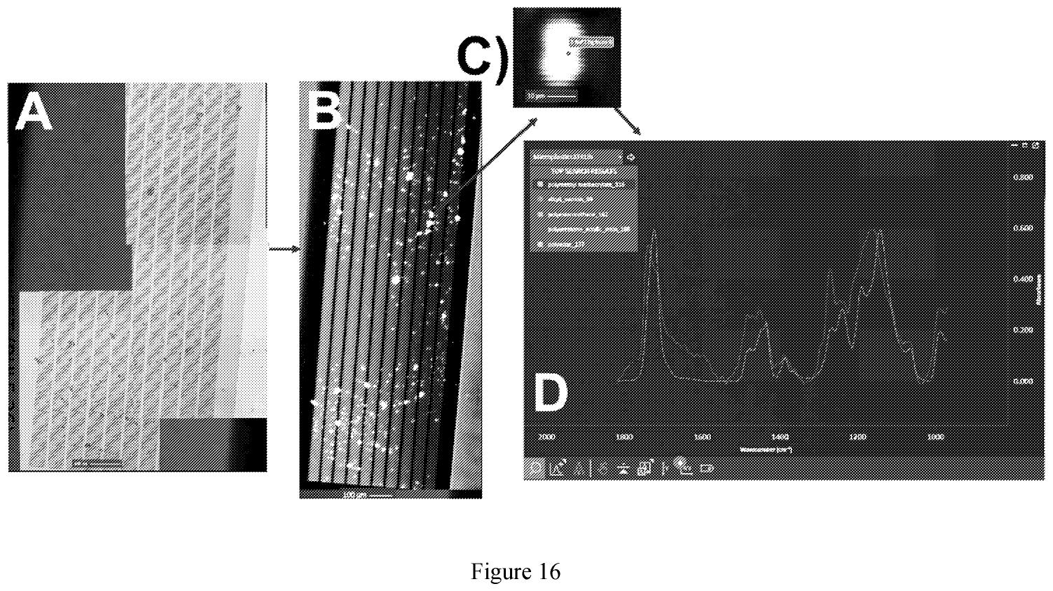

FIG. 16 demonstrates an FTIR microscopy workflow for the identification and quantitation of unknown components (e.g. microplastic particles) captured by the microslit filter from a liquid sample (e.g., a water sample). A similar workflow could be used for another spectroscopy methods for the analytical assay (e.g., Raman or electron dispersive spectroscopy). FIG. 16A shows a region-enhanced composite image of PMMA microparticles retained on a Microslit filter using phase contrast light microscopy. FIG. 16B demonstrates an image of the same membrane and region of interest taken using imaging FTIR. FIG. 16C demonstrates the automated identification of a plastic particle using an algorithm to register and then to identify the particle's composition (example shows a 10 μm diameter polymeric particle). The 8700 LDIR software demonstrating a 10-micron particle). FIG. 16D shows the IR spectra of the 10 μm particle compared to a reference spectrum drawn from a reference library, showing that the particle's composition most closely matches that of PMMA.

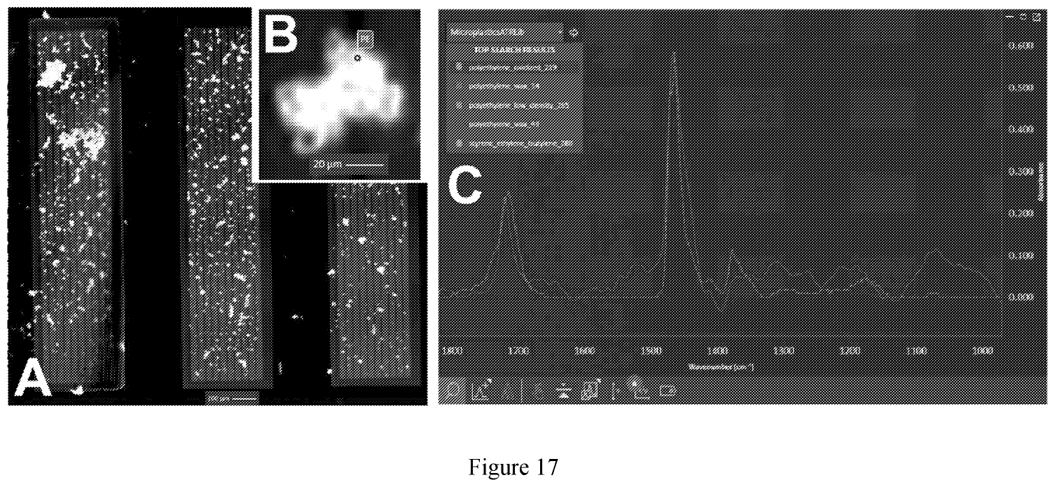

The example shown in FIG. 17 is similar to that shown in the previous figure. FIG. 17A shows a microslit filter with retained PE particles imaged using the 6700 LDIR system in IR mode. FIG. 17B shows a ˜24-micron condensate of multiple PE particles identified by the LDIR software. FIG. 17C shows the best-fit spectra matching that of PE. These data indicate microplastics may be retained selectively by microslit filters, and so retained, can be sized and compositionally analyzed by analytical assays such as FTIR or other similar spectroscopic methods.

### Example 12

This example provides a description of a representative biological component production test using the transmembrane pressure-dependent method of the present disclosure for two types of biological components (e.g., Adenovirus and Maraba virus) and compares the method between a representative microslit filter and a representative polymeric filter.

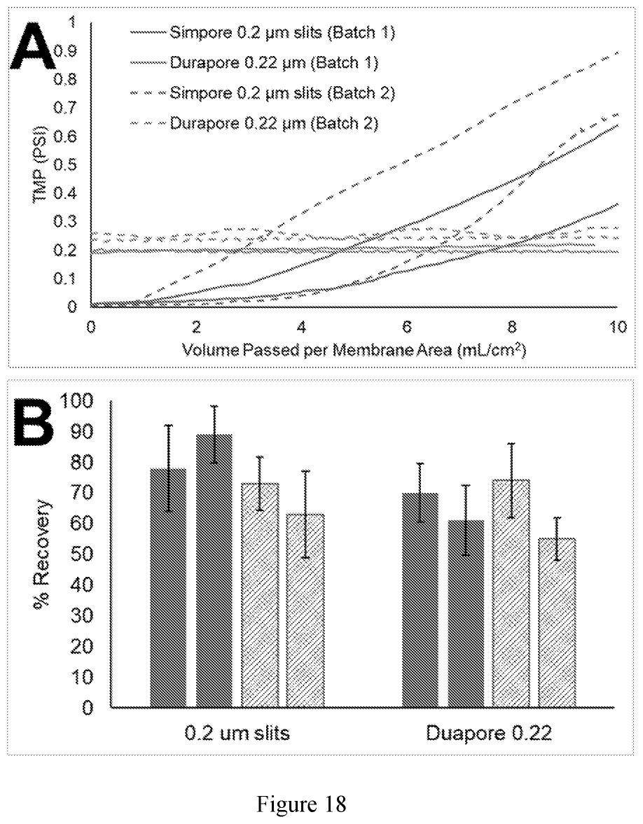

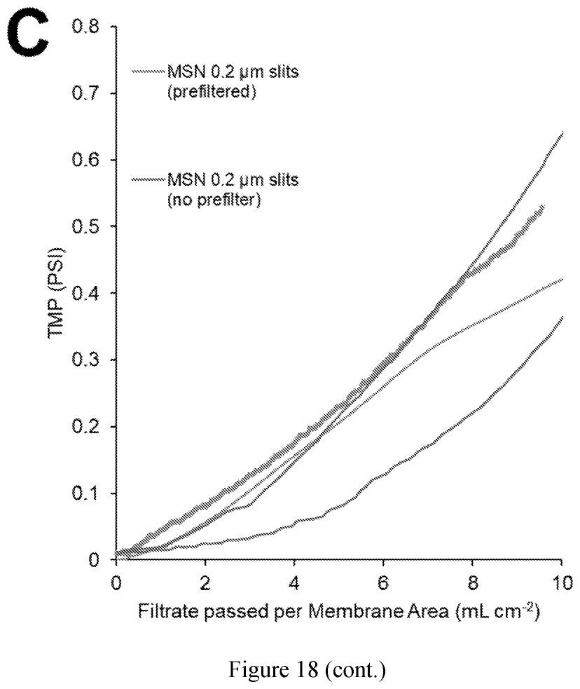

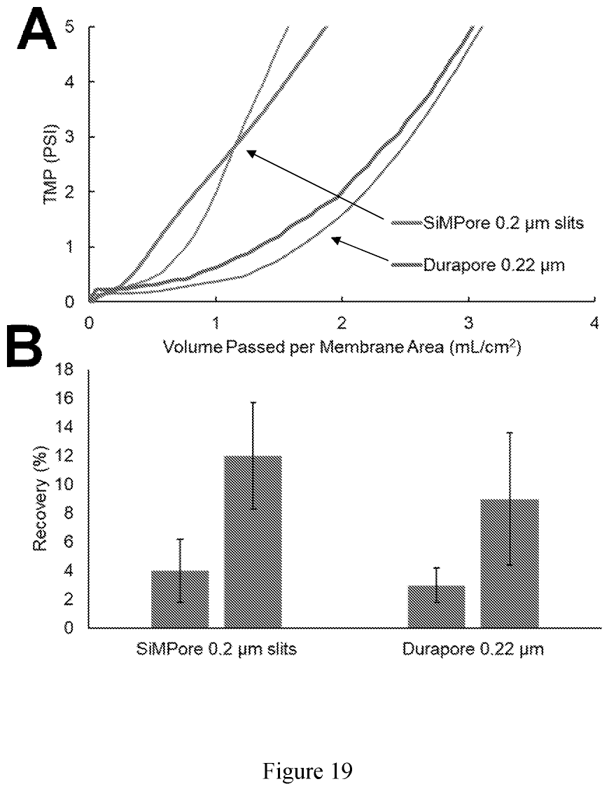

FIGS. 18 and 19 demonstrate filtration of a biological component produced for potential therapeutic applications (e.g., gene therapy, oncolytic viral therapy). Two different batches of adenovirus feedstock were prepared according to the method of Kawka et al (2019) Purification of therapeutic adenoviruses using laterally-fed membrane chromatography. J Memb Sci 579, 351-358. Viral suspension was tittered to therapeutic dosing equivalent by diluting either batch in PBS supplemented with BSA. Feedstocks from both batches were passed through 0.2×10 μm microslit filters.

FIG. 18 compares the filtration of the two adenovirus feedstock preparations when filtered by either 0.2×10 μm microslit filters (FIG. 18A) or conventional 0.2 μm pore size polymeric filters (FIG. 18B; Millipore Durapore™ membranes). Filtration was assessed by the volume permeated and the transmembrane pressure (TMP) profile during filtration. The TMP profiles demonstrate that microslit filters are more sensitive to the size of components in the feedstock, as the faster rise in TMP for microslit filters suggests. A higher virus titer was recovered in the permeate of microslit filters, as measured by plaque forming unit (PFU) assay (FIG. 18B). In all cases, two replicate experiments were performed for each filter type. FIG. 18C shows a pre-treatment of the same Adenovirus feed stock as above using a 0.5×50 micron MicroSlit filter. Upon filtration 0.2×5 micron Microslit filter, the same TMP rise was observed, suggesting that the 0.5×50 micron Microslit filter did not retain any aggregates larger than 0.5 microns from the feedstock supply. These data imply that any aggregates within the feedstock may have an average particle diameter between 200 and 500 nm. This example further demonstrates a serial filtration as described in the present disclosure.

FIG. 19 shows a similar experiment to that shown in the previous figure. In a similar manner to FIG. 18, FIG. 19A shows TMP rise relative to feedstock volume passed per cm\hat{ }2 of filter surface area. In these experiments, the 0.2×10 μm microslit filters showed a rapid increase in TMP, indicating sensitive detection of components in the feedstock that were larger than the slit openings. Similar experiments were performed in parallel using 0.2 pore size Durapore® filters. FIG. 19B shows higher recovery of virus in the permeate from microslit filters.

### Example 13

This example provides a description of a representative biological component production test using the transmembrane pressure-dependent method of the present disclosure for a third type of a biological component (e.g., Brevumondias diminuta bacteria) and compares the method between a representative microslit filter and a representative polymeric filter.

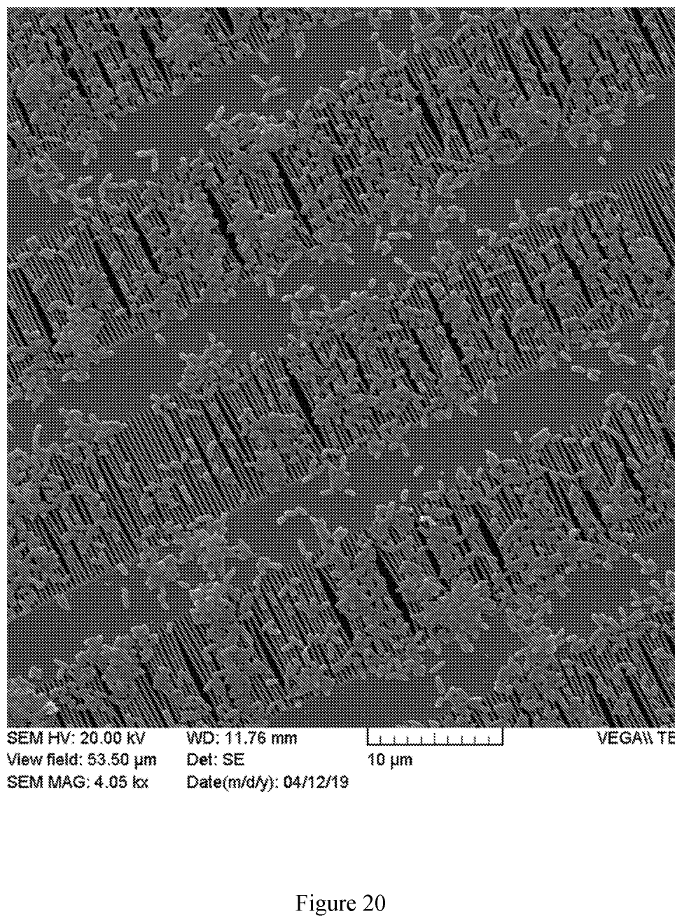

FIG. 20 shows the resulting 0.2×10 microslit filter after filtration of a suspension solution of B. diminuta, using cryo-transmission electron microscopy. Thus, FIG. 20 represents a further example of an ex situ analytical assay. In all related experiments, bacteria were grown on tryptic soy agar plates or as suspension cultures in saline lactose broth or tryptic soy broth. B. diminuta ATCC® 19146 was used throughout. Following 24-hour cultures in tryptic north and saline lactose broth, material dilutions were prepared in saline lactose broth. The filtration apparatus shown in FIG. 13 was used for the related experiments and volume passed and TMP were recorded. The apparatus was wetted with saline lactose broth and an aliquot of permeating broth was recovered for plate-culture assay. B. diminuta suspension was supplied at 2 mL cm−2 min−1. The filtrate was collected, streaked on plates, and cultured for 48 hours, after which plate colonies were counted. All experiments were performed in triplicate. These data show complete retention of bacteria. Moreover, the TMP profiles for 0.2×10 μm microslit filters show a dependence on bacterial concentration in the supplied feedstock, demonstrating that TMP can be used to evaluate the amount of biological components in a liquid sample.

### Example 14

This example provides a fuller description of various membranes and filters represented throughout the present disclosure in the exemplary devices and methods.

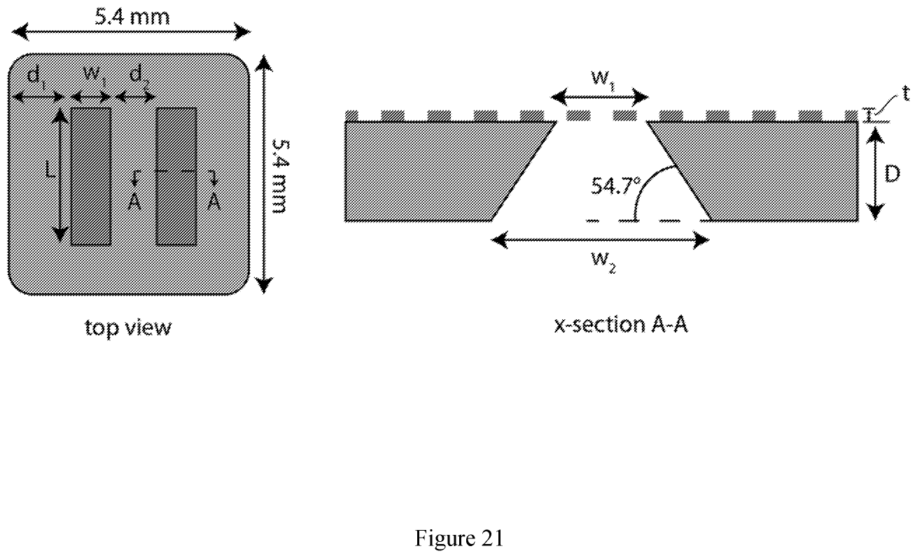

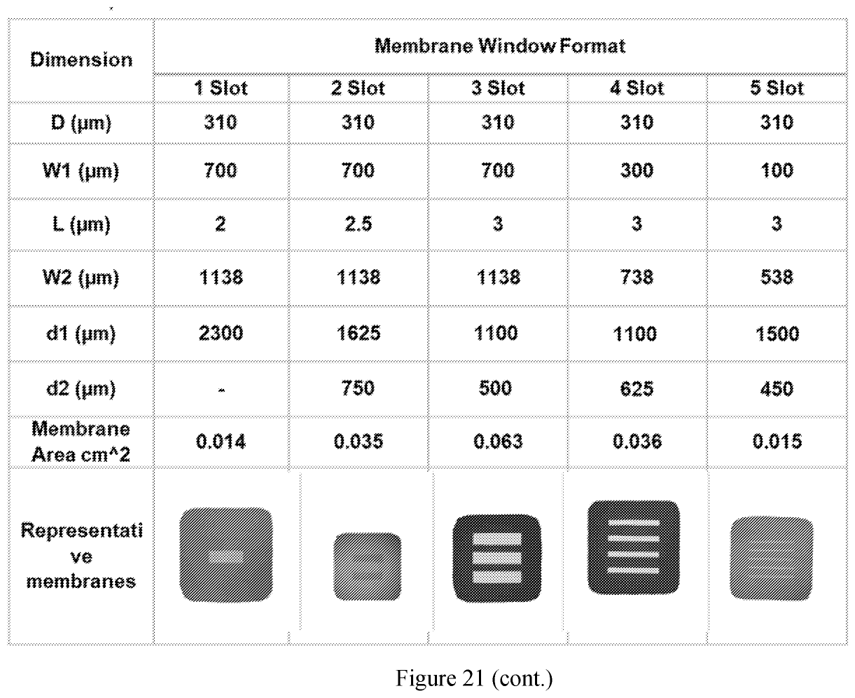

FIG. 21 provides descriptive statistics for various membrane filters routinely produced by silicon-based fabrication processes, including the 5.4×5.4×0.3 mm format used throughout the present disclosure. This format is integrated into the centrifuge spin column fluidic devices; for example, those fluidic devices shown in FIG. 1. Corresponding light microscopy images of the various membrane formats are also shown, with varying amounts of freestanding membrane areas. Data on the mechanical robustness of these varying membrane areas is also provided.

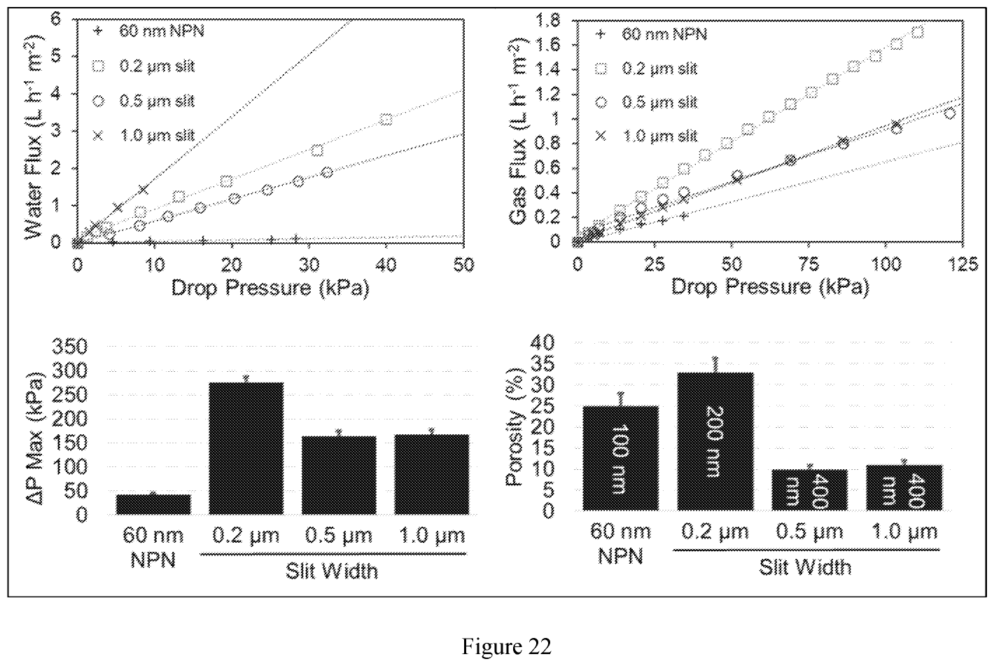

FIG. 22 shows various membrane properties for microslit and nanoporous membrane filter formats, in terms of water and gas permeance, maximum differential pressure tolerance (a.k.a. “burst pressure”), and membrane physical properties (porosity, thickness, and average pore size)

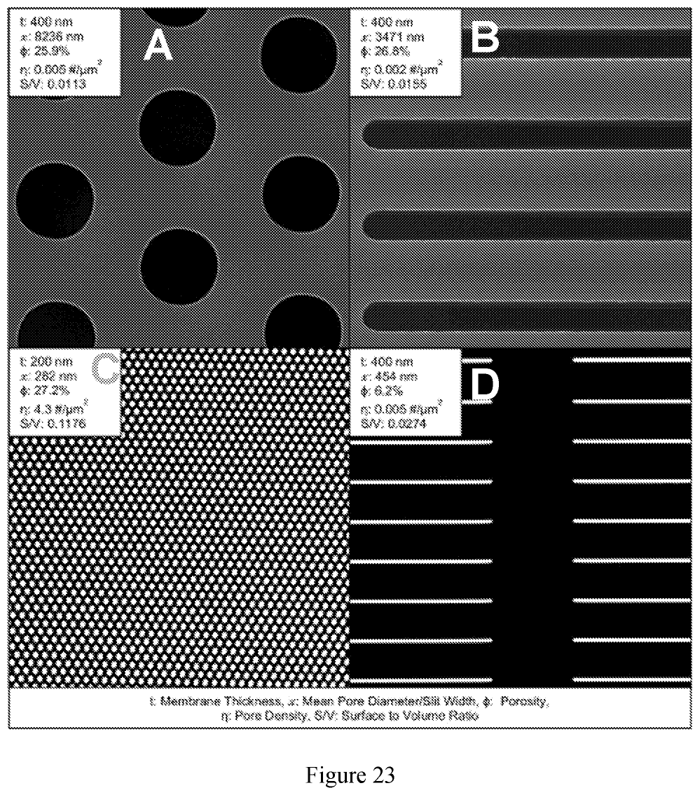

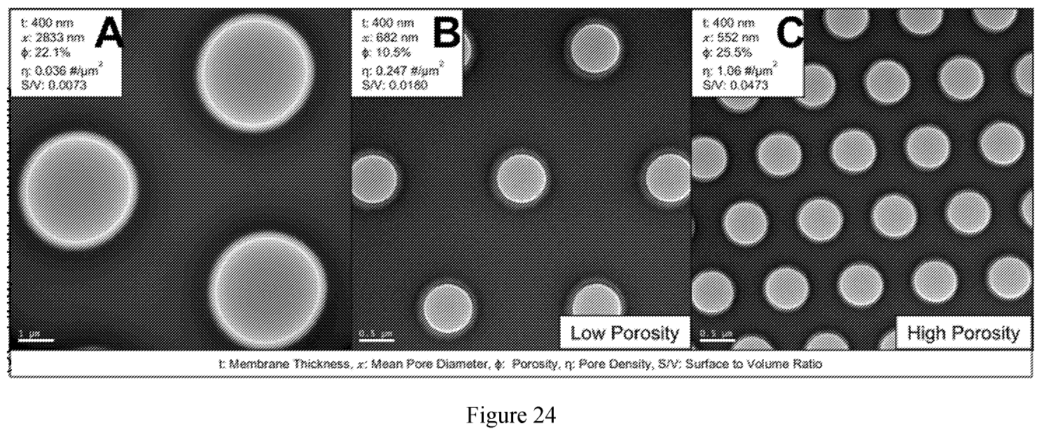

FIGS. 23 and 24 provide examples of microslit and microporous membrane formats, whereby permeability (by way of pore size and porosity) is provided.

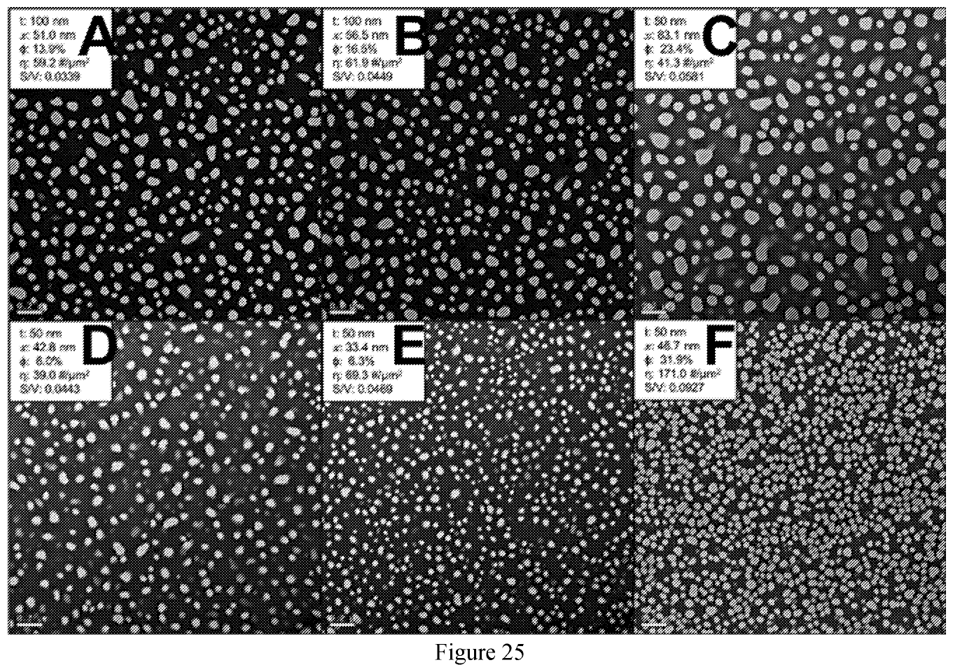

FIG. 25 shows various nanoporous membrane filters with membranes of a various pore diameter and overall pore density

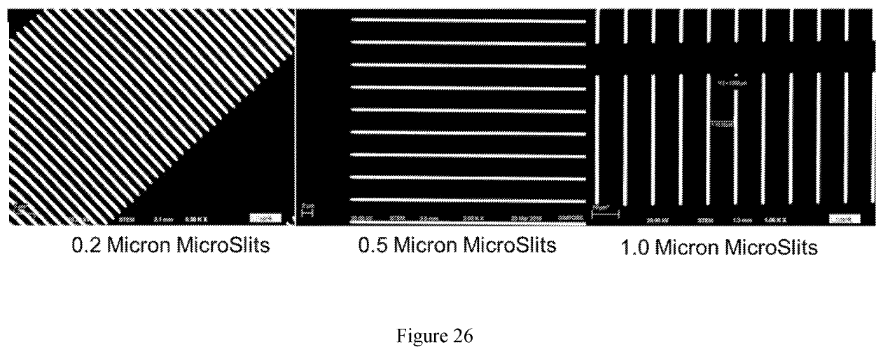

FIG. 26 shows a comparison of 2 scanning electron micrographs for 0.2×10 μm, 0.5×50 μm, and 1×50 μm slit opening microslit filters.

### NON-PATENT CITATIONS

i. Leverett, L. B., Hellums, J. D., Alfrey, C. P., and Lynch, E. C. (1972). Red blood cell damage by shear stress. Biophys J 12, 257-273.
ii. Kawka, K., Madadkar, P., Umatheva, U., Shoaebargh, S., Medina, M. F. C., Lichty, B. D., Ghosh, R., and Latulippe, D. R. (2019). Purification of therapeutic adenoviruses using laterally-fed membrane chromatography. J Memb Sci 579, 351-358.

### PATENT CITATIONS

i. Huff, J. B. et al. Devices and methods for sample analysis. WO2016161402A1.
ii. Miller, B. and Rothberg, L. Method for biomolecular sensing and system thereof. U.S. Pat. No. 7,292,349.
iii. Shestopalov, A., McGrath, J. & Li, X. Methods for depositing a monolayer on a substrate. U.S. Pat. No. 9,089,819.
iv. Striemer, C. C., et al. Methods for Creating Fluidic Cavities by Transmembrane Etching Through Porous Membranes and Structures Made Thereby and Uses of Such Structures. U.S. Pat. No. 62/248,467.
v. Carter, J. A. and Roussie, J. A. Functionalized Silicon Nanomembranes and Uses Thereof. U.S. Pat. Appln. No. 62/614,232.
vi. Striemer, C. C. et al. Nanoporous Silicon Nitride Membranes, and Methods for Making and Using Such Membranes. U.S. Pat. No. 9,789,239.
vii. Striemer, C. C. et al. “Free-Standing Silicon Oxide Membranes, and Methods of Making and Using Same. U.S. Pat. No. 9,945,030.

Although the present disclosure has been described with respect to one or more particular embodiments and/or examples, it will be understood that other embodiments and/or examples of the present disclosure may be made without departing from the scope of the present disclosure.

1. A method comprising:
contacting a liquid sample comprising a plurality of components with a microslit filter, such that one or more components of interest are retained by the microslit filter and undesired components permeate through the microslit filter;
optionally, washing the retained components of interest; and
measuring the quantity of retained components of interest to determine the concentration of the retained components of interest in the liquid sample.

2. The method of claim 1, wherein measuring the quantity of retained components of interest comprises optical imaging, electronic interrogation, optical diffraction, or transmembrane pressure.

3. The method of claim 1, wherein the one or more components of interest comprises a biological component.

4. The method of claim 1, wherein the liquid sample is a biofluid sample.

5. The method of claim 1, wherein the one or more components of interest comprises a non-biological component.

6. The method of claim 1, wherein the liquid sample comprises a food, environmental, or industrial sample.

7. The method of claim 1, wherein the measuring the quantity of retained components of interest is performed as a single instance or as replicate instances.

8. The method of claim 1, wherein the contacting a liquid sample with a microslit filter, such that one or more components of interest are retained by the microslit filter and undesired components permeate through the microslit filter includes gravity flow, hydrostatic pressure, pumping, vacuum, centrifugation, gas pressurization, or tangential flow.

9. The method of claim 1, wherein the contacting a liquid sample with a microslit filter, such that one or more components of interest are retained by the microslit filter and undesired components permeate through the microslit filter occurs at a pressure from 10 Pa to 1.0 kPa.

10. The method of claim 1, further comprising preparing one or more upstream sample.

11. The method of claim 1, further comprising performing one or more first analytical assay on the one or more retained components of interest.

12. The method of claim 1, further comprising adding a binding agent and performing a first analytical assay on the one or more retained components of interest.

13. The method of claim 1, further comprising eluting the one or more retained components of interest and performing a second analytical assay.

14. The method of claim 1, wherein the sample comprises a combination of biological and non-biological components.

15. The method of claim 1, further comprising performing a microplastic particle test on a liquid sample.

16. The method of claim 1, further comprising performing a biological component production test.

17. A method comprising:
contacting a liquid sample comprising a plurality of components with two or more microslit filters, such that two or more populations of components of interest are retained by the two or more microslit filters and undesired components permeate through the two or more microslit filters;
optionally, washing the retained components; and
measuring the quantity of the two or more populations of retained components to determine the concentration of the two or more populations of retained components in the liquid sample.

18. The method of claim 17, wherein measuring the quantity of the two or more populations of retained components to determine the concentration of the two or more populations of retained components in the liquid sample comprises optical imaging, electronic interrogation, or transmembrane pressure.

19. The method of claim 17, wherein the components of interest comprise a biological component, a non-biological component, or combinations thereof.

20. The method of claim 17, wherein the liquid sample is a biofluid sample, a food sample, an environmental sample, an industrial sample, or combinations thereof.

21. The method of claim 17, wherein contacting a liquid sample with two or more microslit filters comprises fluidic contact of the liquid sample with two or more first filtration membranes, two or more fluidic cavities, and two or more second filtration membranes.

22. The method of claim 17, wherein measuring the quantity of the two or more populations of retained components is performed as a single instance or as replicate instances for each component population.

23. The method of claim 17, wherein contacting a liquid sample comprising a plurality of components with two or more microslit filters, such that two or more populations of components of interest are retained by the two or more microslit filters and undesired components permeate through the two or more microslit filters includes gravity flow, hydrostatic pressure, pumping, vacuum, centrifugation, gas pressurization, or tangential flow.

24. The method of claim 17, wherein contacting a liquid sample comprising a plurality of components with two or more microslit filters, such that two or more populations of components of interest are retained by the two or more microslit filters and undesired components permeate through the two or more microslit filters occurs at a pressure from 10 Pa to 1.0 kPa.

25. The method of claim 17, further comprising preparing one or more upstream sample.

26. The method of claim 17, wherein the liquid sample is whole blood and the plurality of components comprise leukocytes, erythrocytes, and platelets.

27. The method of claim 26, further comprising performing a complete blood cell count.

28. The method of claim 17, further comprising performing one or more first analytical assay on the two or more populations of retained components of interest.

29. The method of claim 17, further comprising adding a binding agent and performing a first analytical assay on the two or more populations of retained components of interest.

30. The method of claim 17, wherein the liquid sample is whole blood and the measured components comprise leukocytes, erythrocytes, and platelets, and the method further comprises using binding agents and performing first analytical assays, such that a complete blood cell count with differentiation results.

31. The method of claim 17, further comprising performing a microplastic particle test on the liquid sample.

32. The method of claim 17, further comprising performing a biological component production test.

33. The method of claim 17, further comprising eluting the two or more populations of retained components to perform a second analytical assay.

34. A device comprising one or more microslit filters, wherein each microslit filter comprises a plurality of openings.

35. The device of claim 34, further comprising a first fluidic channel or chamber on a side of the microslit filter and a second fluidic channel or chamber on an opposite side of the microslit filter.

36. The device of claim 34, wherein the microslit filter has a thickness from 50 nm to 25 μm.

37. The device of claim 34, wherein the microslit filter has a porosity from 1% to 75%.

38. The device of claim 34, wherein the openings are cubic prisms, rectangular prisms, or trapezoids.

39. The device of claim 34, wherein the openings have a width from 0.5 μm to 15 μm and a length from 5 μm to 100 μm.

40. The device of claim 34, wherein the openings have an aspect ratio from 1:0.33 to 1:200.

41. The device of claim 34, wherein the at least one microslit filter further comprises at least one filtration membrane, one fluidic cavity, and one second filtration membrane, wherein the one fluidic cavity is an intervening fluidic cavity.

42. The device of claim 34, wherein the microslit filter further comprises a metal coating.

43. The device of claim 34, wherein the microslit filter is 400 nm thick and has 17% porosity, wherein the openings are at least 8 μm in width and 50 μm in length and have an aspect ratio of at least 1:6.25.

44. The device of claim 34, wherein the microslit filter is 400 nm thick and has 9% porosity, wherein the openings are 1 μm in width and 50 μm in length and have an aspect ratio of 1:50.

45. The device of claim 34, wherein the microslit filter is 400 nm thick and has 9% porosity, wherein the openings are 0.5 μm in width and 50 μm in length and have an aspect ratio of 1:100.

46. The device of claim 34, further comprising one or more non-fouling surfaces.

47. The device of claim 34, wherein the microslit filter is further configured to increase interactions between one or more retained components and the microslit filters.

48. The device of claim 34, further comprising a light source and a detector configured to record optical signals of an optical modality.

49. The device of claim 48, wherein the optical modality is an intensity of a diffraction pattern.

50. The device of claim 48, wherein the light source and the detector are configured to record optical signals of an optical modality by performing Fourier-transformed infrared spectroscopy or Raman spectroscopy.

51. The device of claim 34, further comprising an electrical interrogator configured to record an electrical property.

52. The device of claim 51, wherein the electrical interrogator comprises a voltmeter, or one or more pairs of electrodes.

53. The device of claim 51, wherein the electrical interrogator comprises a function generator.

54. The device of claim 51, wherein the electrical property is conductivity, resistance, impedance, current, or voltage.

55. The device of claim 34, further comprising one or more pressure sensors configured to record trans-membrane pressure of a pressure-dependent modality.

56. The device of claim 55, wherein the pressure-dependent modality is a trans-membrane pressure drop.

57. The device of claim 34, further comprising a flow meter.

58. The device of claim 34, further comprising at least one sample microslit filter and at least one reference microslit filter.

59. The device of claim 34, further comprising at least one additional microslit filter configured to perform an upstream sample preparation.

60. The device of claim 34, further comprising a fouling sensor configured to detect a level of fouling on at least one portion of the microslit filter.

61. The device of claim 34, further comprising an optical imaging device configured to record an optical imaging modality.

62. The device of claim 61, wherein the optical imaging modality is surface plasmon resonance, plasmon-enhanced fluorescence, surface, enhanced fluorescence, or surface-enhanced Raman spectroscopy.

63. The device of claim 34, wherein the device is a fluidic device.

64. A kit comprising one or more device of claim 34 and/or one or more reagents.

65. The kit of claim 64, wherein the kit further comprises instructions for use of the one or more device and/or one or more reagents.

66. The kit of claim 64, wherein the kit further comprises instructions for:
contacting a liquid sample comprising a plurality of components with a microslit filter, such that one or more components of interest are retained by the microslit filter and undesired components permeate through the microslit filter;
optionally, washing the retained components of interest; and
measuring the quantity of retained components of interest to determine the concentration of the retained components of interest in the liquid sample.

67. The kit of claim 64, wherein the kit further comprises instructions for:
contacting a liquid sample comprising a plurality of components with two or more microslit filters, such that two or more populations of components of interest are retained by the two or more microslit filters and undesired components permeate through the two or more microslit filters;
optionally, washing the retained components; and
measuring the quantity of the two or more populations of retained components to determine the concentration of the two or more populations of retained components in the liquid sample.

68. The kit of claim 64, wherein the one or more reagents are selected from binding agents, buffers, solutions, and/or reagents for enzymes and the like, model components in solution (of known concentration), and combinations thereof.

69. The kit of claim 64, wherein the binding agents are selected from affinity moieties, detection agents, and combinations thereof.

70. The kit of claim 64, wherein the kit further comprises a light source and/or a detector.

71. The kit of claim 64, wherein the kit further comprises a flow meter.

72. The kit of claim 64, wherein the kit further comprises a voltmeter, one or more pair of electrodes, and optionally, a function generator.

73. The kit of claim 64, further comprising one or more pressure sensors.

74. The kit of claim 64, further comprising a signal processing algorithm for optical, electronic, and/or pressure signals.

75. The kit of claim 64, further comprising one or more algorithms for calculating concentration of components from a liquid sample.

76. The kit of claim 64, further comprising one or more algorithms for registering retained components on microslit filters and determine the size and/or composition of such components.
